(19) United States
(12) Patent Application Publication
(10) Pub. No.: US 2008/0279901 A1
(43) Pub. Date: Nov. 13, 2008

(54) Composition comprising encapsulated silicone compounds

(75) Inventors: Fanny Prigent, Ceyrat (FR); Jean-Thierry Simmonet, Cachan (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/003,026

(22) Filed: Dec. 19, 2007

Related U.S. Application Data
(60) Provisional application No. 60/883,175, filed on Jan. 3, 2007.

(30) Foreign Application Priority Data
Dec. 20, 2006 (FR) 06 55681

(51) Int. Cl. A61K 8/11; A61K 8/89; A61Q 1/00; A61Q 15/00

(52) U.S. Cl. 424/401; 424/78.02; 424/65

(57) ABSTRACT

The present invention relates to a cosmetic composition intended for the care and/or make-up of keratinous substance(s) comprising, in a physiologically acceptable medium, at least one compound X, one compound Y and at least one catalyst, with at least one of the compounds X or Y being a polyorganosiloxane, and said compounds X and Y being capable of reacting together by a hydrosilylation reaction in the presence of a catalyst, with at least one compound from the compounds X and Y being present in said composition in an encapsulated form, said catalyst being combined with at least one of said encapsulated compound X or Y.

This non provisional application claims the benefit of French Application No. 06 55681 filed on Dec. 20, 2006 and U.S. Provisional Application No. 60/883,175 filed on Jan. 3, 2007.

The present invention relates to a cosmetic composition comprising at least one compound X and one compound Y capable of reacting together, where appropriate in the presence of a catalyst or a peroxide, at least one of the compounds being a polyorganosiloxane and at least one of the compounds being in an encapsulated form.

The compositions considered according to the invention are more particularly intended for the care and/or make-up of keratinous substances and especially the skin, lips and integuments.

Generally, the cosmetic compositions are intended to provide an aesthetic effect and this aesthetic effect is generally obtained by the formation of a film of make-up and/or care product on the support in question such as, for example, the face, lips, eyelashes and nails.

For obvious reasons, optimization of the qualities of comfort, durability over time and/or non-transfer of these films is a constant concern in the cosmetics field.

It is known that certain systems comprising silicone compounds are capable, by simple contacting of these compounds, where appropriate in the presence of a catalyst or a peroxide, of producing polymeric silicone films. Thus, compounds known as compound X and compound Y, such as defined below, prove capable of polymerizing in situ, at atmospheric pressure and at ambient temperature and of forming films that are advantageously biocompatible, non-tacky, slightly opalescent or even peelable. Such systems are, in particular, partly described in documents WO 01/96450 and GB 2 407 496. However, considering the high reactivity of these compounds, it is essential to package them separately to prevent the premature formation of a film.

Consequently, the use of these systems in the care and/or make-up field imposes a mode of packaging or even of application similar to that of the double-action product in order to guarantee that the mixing of the two compounds that form the system, carried out where appropriate in the presence of a catalyst or a peroxide, only takes place in contact with the support in question or is only produced extemporaneously just before its application to the support.

It is clear that this need to package the two compounds separately, or where appropriate the two compounds and the catalyst or the peroxide separately, represents a constraint for both the compounder and the user which it would be desirable to be free of.

The present invention aims specifically to overcome this constraint.

The inventors have thus observed that it proves possible to formulate such a system as a single composition without however prejudicing the reactivity of its two components.

More specifically, the present invention relates, according to one of its aspects, to a cosmetic composition intended for the care and/or make-up of keratinous substance(s) comprising, in a physiologically acceptable medium, at least one compound X and one compound Y and optionally at least one catalyst or peroxide, with at least one of the compounds X or Y being a polyorganosiloxane, and said compounds X and Y being capable of reacting together by a hydrosilylation reaction in the presence of a catalyst, or by a condensation reaction, or by a crosslinking reaction in the presence of peroxide, with at least one compound from the compounds X, Y, and the catalyst or peroxide when they are present, being present in said composition in an encapsulated form, and in which, when compounds X and Y are capable of reacting together by a hydrosilylation reaction in the presence of a catalyst, said catalyst is combined with at least one of the encapsulated compounds X or Y.

In the sense of the invention, it is understood that the two compounds X and Y are not present in combination with the catalyst in one and the same capsule. This is because, with regard to their reactivity, only their reaction product would then be present within the composition. However, within the context of the invention and as emerges from the definition of the compositions, the two compounds X and Y may be set apart from one another.

According to one preferred variant of the invention, the two compounds X and Y are both present in separate encapsulated forms.

More particularly, the encapsulated form is a nanocapsule of core/shell type of which the core of lipophilic nature is formed completely or partly either from at least one compound X, or from at least one compound Y, or from an oil containing at least one catalyst or peroxide if necessary for the interaction of the compound X and of the compound Y present in said composition, said catalyst being encapsulated with the compound X or the compound Y.

According to one advantageous embodiment variant, the shell of the nanocapsules comprises at least one polymer chosen from polycaprolactones.

According to yet another of its aspects, the present invention relates to the use of an encapsulated form of at least one compound X, of an encapsulated form of at least one compound Y, and/or of an encapsulated form of at least one catalyst or of a peroxide if necessary for the interaction of a compound X with a compound Y, for preparing a composition for the care and/or make-up of keratinous substance(s) comprising a physiologically acceptable medium and intended to form, after application and drying on said keratinous substance, a polymeric film derived from the reaction of a compound X and a compound Y according to a reaction of hydrosilylation type in the presence of a catalyst, or a condensation reaction, or a crosslinking reaction in the presence of peroxide, with at least one of the compounds X or Y being a polyorganosiloxane.

According to one advantageous embodiment variant, when the catalyst is present, it is contained in the same capsules as those containing one of the compounds X or the compound Y on condition that the compounds X, Y and the catalyst are not encapsulated together in common capsules.

Thus, according to one embodiment variant, the catalyst is not encapsulated in capsules which will be specific to it, that is to say in a form separate from the compounds X and/or Y.

The invention moreover targets a cosmetic coating method for the care and/or make-up of keratinous substance(s) comprising at least the application on said keratinous substance of a mixture of compounds X and Y capable of reacting together by a hydrosilylation reaction in the presence of a catalyst, or by a condensation reaction, or by a crosslinking reaction in the presence of a peroxide with at least one of the compounds X or Y being a polyorganosiloxane, said mixture being derived from a cosmetic composition comprising, in a physiologically acceptable medium, at least one such compound X and one such compound Y and optionally at least one catalyst or a peroxide, and in which at least one compound from the compounds X, Y and the catalyst or peroxide if present, is present in said composition in an encapsulated form.

According to one embodiment, the composition may be such as defined previously.

The mixture of compounds X and Y may be obtained either extemporaneously before application to said keratinous substance, or at the time of application of said composition to said keratinous substance.

In the sense of the invention, it is to be understood that the mixture thus formed comprises compounds X and/or Y in a form that has not yet reacted and not exclusively in the form of their reaction product by hydrosilylation, by polycondensation and/or by crosslinking in the presence of a peroxide.

Thus, formation of the reaction product according to the invention can either be carried out directly on the surface of the keratinous substance that is to be treated, or initiated just before application by extemporaneous mixing of compounds X and Y in conditions favourable for their interaction, formation of the reaction product being in the latter case finalized on the surface of the keratinous substance.

For obvious reasons, and in view of the great reactivity of compounds X and/or Y, it is in fact necessary that their application should be carried out in conditions that are favourable for the manageability of the composition containing it (or them) notably with respect to its spreading, for example. The method according to the invention therefore employs a composition containing compounds X and Y, and therefore not congealed in the form of the expected final film resulting from reaction of all of X and/or of all of Y.

As it emerges from the examples that appear below, the inventors have observed that the compositions such as defined previously prove stable over time and remain effective for forming a polymeric silicone film. When these compositions are spread in the form of a film over a support, for example a keratinous substance, the encapsulated form or forms break up on drying and the compounds X and Y then brought into contact, where appropriate, in the presence of a catalyst, react together to form a film that is peelable, non-tacky and slightly opalescent. Advantageously, the compositions according to the invention make it possible to postpone the reaction of the compounds X and Y which only takes place at the time when the composition applied in the form of a film to the support in question dries.

Encapsulated Form

In the cosmetic and pharmaceutical fields, it is common to formulate active agents in encapsulated forms out of concern, in particular, of guaranteeing them an increased stability within a composition.

More particularly considered within the context of the present invention are the particular forms of core/shell type also known as microcapsules or nanocapsules, of which the shell is of polymeric nature and the core is of lipophilic nature and more particularly contains at least one oil. The compounds X or Y combined where appropriate with a catalyst, is encapsulated within this lipophilic core.

Numerous techniques are currently available for preparing this type of microcapsules or nanocapsules.

The most conventional approach rests on the emulsification technique and involves the simultaneous synthesis of the polymeric shell by crosslinking the corresponding monomers in the presence of the lipophilic active agent to be encapsulated. For example, document FR 1 583 243 describes a process for manufacturing microcapsules that contain droplets of oil of which the size varies from 0.1 to several microns and which uses chemical crosslinking agents. This technique is also illustrated in document WO 02/09862. Application WO 93/08908 uses this same technique for preparing nanocapsules of which the shell derives from the crosslinking of proteins. Application WO 02/051536 itself describes suspensions of particles having a size between 70 nm and 5 μm, obtained by crosslinking of monomers, this crosslinking being initiated by exposing the suspension to UV radiation.

These techniques may be applied for preparing encapsulated forms of compounds X and Y, encapsulated where appropriate with a catalyst.

However, according to one preferred mode, the encapsulated forms considered according to the invention, are nanocapsules and are especially obtained by a technique known as solvent displacement which consists in dissolving the compounds X or Y, and where appropriate the catalyst or the peroxide, and the polymer having to form the envelope of the nanocapsule, within a solvent. The formation of nanocapsules is initiated by addition to this solution, with stirring, of a subsidiary solvent incapable of dissolving the polymer, the compound X or Y, the catalyst and the peroxide but, on the other hand, that is miscible with the first solvent. This technique is especially illustrated in documents EP 274 961 and EP 1 552 820.

According to one preferred variant, the technique used is more particularly that described in document EP 1 552 820 which involves the formation of a pre-emulsion.

More precisely, this process comprises:
a) the formation of a single phase liquid organic phase comprising at least:
   an organic solvent, having a boiling point less than or equal to 100° C.;
   a polymer soluble in said solvent medium;
   an oil containing at least one compound X or Y or a catalyst or peroxide if necessary for the interaction of the compound X with the compound Y jointly present; and
   a non-ionic surfactant;
b) the preparation of an aqueous phase containing at least one non-ionic surfactant and where appropriate an ionic surfactant;
c) the dispersion of the organic phase (a) in the aqueous phase (b) so as to obtain a pre-emulsion; and
d) the submission of the pre-emulsion obtained in (c) to homogenization in order to obtain the formation of a dispersion of nanocapsules having an average size less than or equal to 1 μm and containing in their lipophilic core at least one compound X or Y or the catalyst or peroxide necessary for the interaction of the compound X with the compound Y.

A similar method may also enable the formation of a dispersion of nanocapsules containing in their lipophilic core at least one compound X or Y encapsulated with at least one catalyst necessary for the interaction of the compound X with the compound Y.

The particles may be recovered in the form of an aqueous dispersion after evaporation of the organic phase and where appropriate of some of the water and used in this form for preparing cosmetic compositions according to the invention.

The shell of the nanocapsules used according to the invention is of polymeric nature.

More particularly, the shell of the nanocapsules used according to the invention is of polymeric nature that is non-crosslinked, water-insoluble and insoluble in the core of the nanocapsules.

Generally, all polymers, of natural or synthetic origin, soluble in a solvent that is immiscible with water and especially those having a melting point below the boiling point of water at atmospheric pressure (100° C.) may be suitable.

These polymers may be biodegradable, such as for example polyesters, or may not be biodegradable.

By way of illustration of polymers that are suitable for the invention, mention may especially be made of:
C₂-C₁₂ and, in particular, C₂-C₆, alkylcyanoacrylate polymers with the alkyl radical possibly, in particular, chosen from ethyl, n-butyl, hexyl, isobutyl and isohexyl radicals;
polymers formed by poly(L-lactides), poly(DL-lactides), polyglycolides and the corresponding copolymers, such as DL-lactide/glycolide copolymers, glycolide/caprolactone copolymers and the like;
polycaprolactones, such as the polycaprolactones having a melting point that varies from 40 to 70° C. and that have a molecular weight between 2 000 and 100 000, such as for example those sold by Solvay under the commercial references CAPA 6806 (MW of 80000), CAPA 6100 (MW of 10000), CAPA 6506 (MW 50000), CAPA 6250 (MW of 25000), CAPA 2803 (MW of 8000), CAPA 2403 D (MW of 4000);
polymers of 3-hydroxybutyric acid;
vinyl chloride/vinyl acetate copolymers, for example those sold under the name RHODOPAS AX 8515® by Rhone Poulenc;
methacrylic acid and ester copolymers, especially copolymers of methacrylic acid and methacrylic acid ester, for example those sold under the name EUDRAGIT L 100® by Rohm Pharma;
polyvinyl acetophthalate;
cellulose acetophthalate;
polyvinyl pyrrolidone/vinyl acetate copolymer;
polyethylene vinyl acetates;
polyacrylonitriles;
polyacrylamides;
polyethylene glycols;
poly(hydroxy(C₁ to C₄)alkyl methacrylate) and in particular poly(hydroxyethyl methacrylate);
derivatives of cellulose such as esters of cellulose and of at least one C₁ to C₄ carboxylic acid, especially mixed cellulose esters of two types of carboxylic acids;
polystyrene and styrene/maleic anhydride copolymers, styrene/acrylic acid copolymers, styrene-ethylene/butylene-styrene block terpolymers, styrene-ethylene/-propylene-styrene block terpolymers;
styrene alkyl alcohol oligomers;
ethylene/vinyl acetate/maleic anhydride terpolymers;
polyamides;
polyethylenes;
polypropylenes;
organopolysiloxanes including polydimethylsiloxanes;
polyalkylene adipates which encompass both homopolymers of adipic acid and of an alkanediol and linear or branched copolymers of poly(ester/ether) type, obtained from adipic acid and from one or more alkanediols and/or etherdiols and/or triols; the alkanediols used for preparing said polyalkylene adipates may be C₂-C₆ alkanediols having a linear or branched chain chosen from ethylene glycol, propylene glycol, 1,3-propanediol, 1,4-butanediol, 1,5-pentanediol, 1,6-hexanediol and neopentyl glycol. The etherdiols are di-, tri- or tetra-(C₂-C₄ alkylene) glycols such as diethylene glycol, triethylene glycol, tetraethylene glycol, dipropylene glycol, tripropylene glycol, tetrapropylene glycol, dibutylene glycol, tributylene glycol or tetrabutylene glycol; by way of example, mention may more particularly be made of the FOMREZ® products sold by Witco and the polyethylene adipates from Scientific Polymer Products; mention may also be made, by way of example, of EASTAR BIO® from Eastman Chemical (polytetramethylene adipate-co-terephthalate) and ECOFLEX F BX 7011® from BASF (1,4-butanediol/terephthalic acid/adipic acid terpolymer);
polyester polyols obtained by polycondensation of an aliphatic dicarboxylic acid with at least two alkanediols or with at least one alkanediol and at least one hydroxy-alkyl alkanediol with the aliphatic dicarboxylic acid possibly being adipic acid (1,6-hexanedioic acid), such polymers are especially described in document FR 2 836 381;
polysilsesquioxane silicone polymers, especially a polyalkylsilsesquioxane of formula: (R—SiO_{3/2})ₓ in which R represents a linear, branched or cyclic, saturated or unsaturated hydrocarbon-based radical; for example of the —CₙH₂ₙ₊₁ type with n being an integer ranging from 1 to 20, especially a methyl, ethyl, propyl, butyl, pentyl, hexyl, heptyl, octyl, nonyl, decyl, dodecyl, tridecyl, tetradecyl, hexadecyl, octadecyl and eicosyl radical; or else an aryl group, especially a phenyl or tolyl group; a cycloalkyl, especially a cyclobutyl, cyclopentyl or cyclohexyl group; an alkenyl group, especially a vinyl or allyl group; an aralkyl group, especially a 2-phenylethyl or benzyl group; R possibly also comprising 1 or more halogen atoms, especially fluorine or chlorine; preferably R is a methyl, ethyl, propyl or phenyl radical; and x is the number of units, and may be between 1 and 10, especially 1 to 4; by way of example mention may be made of BELSIL PMS MK® from Wacker or RESIN KR 220® from Shin Etsu;
dendritic polyesters having a terminal hydroxyl functional group especially those described in document FR 2 790 405;
polymers that are water-dispersible but nevertheless soluble in solvents that are immiscible with water, such as for example: polyesters, polyesteramides, polyurethanes and vinyl copolymers bearing sulphonic and/or carboxylic acid functional groups, and in particular those described in document FR 2 787 729;
block copolymers that are insoluble in water at ambient temperature and solid at ambient temperature, having at least one block of one of the preceding polymers; and
mixtures thereof.

According to one exemplary embodiment, the shell of the nanocapsules may comprise at least one polymer chosen from the group consisting of —C₂-C₁₂ alkyl cyanoacrylate polymers; poly-L-lactides, poly-DL-lactides, polyglycolides and the corresponding copolymers;
polycaprolactones;
polymers of 3-hydroxybutyric acid;
copolymers of vinyl chloride and vinyl acetate;
polyvinyl acetophthalate;
cellulose acetophthalate;
polyvinylpyrrolidone-vinyl acetate copolymer;
polyethylenevinyl acetates;
copolymers of methacrylic acid and methacrylic ester;
polyacrylonitriles;
polyacrylamides;
polyethylene glycols;
poly(C₁ to C₄ hydroxyalkyl methacrylate);
cellulose derivatives;
polystyrene and its copolymers;
styrene alkylalcohol oligomers;
terpolymers of ethylene, vinyl acetate and maleic anhydride;
polyamides;
polyethylenes;
polypropylenes;
organopolysiloxanes;
poly(alkylene adipate);
polyol polyesters;
polysilsesquioxane silicone polymers;
dendritic polyesters with a hydroxyl terminal function; and
mixtures thereof.

These polymers or copolymers may have a weight-average molecular weight between 1000 and 500 000 and in particular between 1500 and 100 000.

The polymer of the shell of the nanocapsules may especially be chosen from:
polycaprolactones;
polyvinyl acetophthalate;
cellulose acetophthalate;
methacrylic acid and ester copolymers;
cellulose derivatives;
polystyrene and its copolymers;
polyamides;
organopolysiloxanes;
polyalkylene adipates; and
mixtures thereof.

The following are most particularly suitable for the invention: polyalkylene adipate, organopolysiloxanes, polycaprolactones, cellulose acetophthalate, cellulose acetobutyrate, cellulose esters, polystyrene, and its derivatives.

Polycaprolactones are preferred.

Of course, a person skilled in the art is able, on the basis of his knowledge, to adjust the molecular weight of the polymer selected with respect to its concentration in the solvent so as to have a mixture viscosity compatible with satisfactory emulsification.

With regard to the lipophilic core, it contains the compound X, the compound Y, or the catalyst or peroxide formulated in at least one oil if necessary for the interaction of the compound X and of the compound Y, or else the catalyst encapsulated with the compound X and/or Y, on the condition that the compounds X, Y and the catalyst are not combined together in one and the same capsule.

In the sense of the present invention, the term “oil” denotes all natural, vegetable or animal, or synthetic oily substances that are liquid from 40° C., that may or may not have one or more recognized biological activities and that are insoluble in water (less than 2 wt % at ambient temperature).

According to one embodiment, the nanocapsules according to the invention have an average diameter (or “size”) of less than 1 μm, preferably less than or equal to 500 nm, for example between 200 and 500 nm, preferably less than or equal to 350 nm, for example between 200 and 350 nm.

This diameter may especially be controlled according to a measurement technique resting on dynamic light scattering with, for example, a BI90+® machine from Broohaven Instrument or a Malvern Mastersizer 2000 (adapted for Ø greater than 2 μm).

According to one variant of the invention, the encapsulated forms of compounds X or compounds Y and/or of a catalyst may be coated with a lamellar phase.

The expression “lamellar phase” (phase D according to EKWALL) is understood to mean a liquid crystal phase with plane symmetry, comprising several amphiphilic bilayers arranged in parallel and separated by a liquid medium which is generally water.

A more precise definition of this expression is given in Ekwall (1968), Adv. Liq. Cryst. (Brown G. H., Ed.), Chap. 1, 14. This phase has a characteristic texture under a polarized light microscope, a more precise description of which can be found in Roservear (1968), JSCC, 19, 581 and in Lachampt et Vila (1969), Revue Française des Corps Gras [French Review of Fatty Substances], No. 2, 87-111.

It is often desirable or necessary to provide the nanocapsules with a so-called “lamellar” coating. This is a structure organized into one or more lipid sheets each formed from a bilayer of amphiphilic molecules similar to that of biological membranes. The polymeric envelope of the nanocapsules according to the invention may thus be surrounded by a lamellar coating having a structure organized into one or more sheets each composed of a double layer of amphiphilic molecules forming a coating agent. This coating, besides its role of adjusting the size of the nanocapsules, improves the sealing of the nanocapsules with respect to a leak of the active agent towards another lipid phase of the composition.

The coating agents are surfactants having a hydrophobic nature, that are soluble in the organic phase used in the method described above and which are capable, in the presence of water, of forming the lipid bilayers described above.

In the method for encapsulating active principles, used by the Applicant, this coating agent is dissolved in the organic phase containing the polymer and the lipid phase.

By way of example of such coating agents, mention may be made of phospholipids such as lecithin as described in document EP-A-447 318; certain polycondensates of ethylene oxide and of propylene oxide, such as the products sold under the name PLURONIC® by BASF such as PLURONIC® L121 or under the name SYNPERONIC® by ICI; or the silicone surfactants (silicones comprising at least one oxyethylenated and/or oxypropylenated chain) capable of forming lamellar structures, such as those described in documents U.S. Pat. No. 5,364,633 and U.S. Pat. No. 5,411,744 and used in Patent Application FR-A 2 742 677, for example those sold by Dow Corning under the names DC 5329, DC 7439-146, DC 2-5695 and Q4-3667; and mixtures thereof.

The lamellar phase may also comprise an ionic amphiphilic lipid. The latter may be chosen from anionic lipids and cationic lipids.

The amphiphilic lipids suitable for use in the invention may be:
neutralized anionic lipids, such as for example the disodium salt of N-stearoyl L-glutamic acid sold under the name Acylglutamate HS21 by Ajinomoto;
amphoteric lipids, preferably phospholipids; and
cationic amphiphilic lipids, which may be used as ionic amphiphilic lipids, may more particularly be chosen from the group formed by quaternary ammonium salts, fatty amines and salts thereof.

As regards the operating procedure for preparing nanocapsules according to the invention, a person skilled in the art could refer, in particular, to the teaching of the aforementioned document EP 1 552 820. The choice of surfactants thus requires that the implementation of the process makes use of the knowledge of the person skilled in the art.

Compounds X and Y

Silicone compound means a polyorganosiloxane compound, i.e. comprising at least two organosiloxane units, for example at least 5 organosiloxane units, notably at least 10 organosiloxane units. According to a particular embodiment, at least one of compounds X and Y, or compounds X and compounds Y are silicone compounds. Compounds X and Y can be aminated or non-aminated.

According to another embodiment, at least one of compounds X and Y is a polymer whose main chain is formed primarily of organosiloxane units. Among the silicone compounds mentioned below, some may display both film-forming and adhesive properties, depending for example on their proportion of silicone or depending on whether they are used mixed with a particular additive. It is therefore possible to adjust the film-forming properties or the adhesive properties of said compounds according to the proposed use, which is the case in particular for the so-called “room temperature vulcanization” reactive elastomeric silicones.

Compounds X and Y can react with each other at a temperature varying between room temperature and 180° C. Advantageously, compounds X and Y are capable of reacting together at room temperature (20±5° C.) and atmospheric pressure, or advantageously in the presence of a catalyst, by a hydrosilylation reaction or a condensation reaction, or a crosslinking reaction in the presence of a peroxide.

Polar Groups

According to a particular embodiment, at least one of compounds X and Y, for example compound X, bears at least one polar group that is able to form at least one hydrogen bond with keratinous substances.

By polar group, we mean a group having carbon atoms and hydrogen atoms in its chemical structure and at least one heteroatom (such as O, N, S and P), such that said group is able to establish at least one hydrogen bond with keratinous substances.

Compounds bearing at least one group that can form a hydrogen bond are particularly advantageous, as they endow the compositions containing them with better adherence on keratinous substances.

The polar group or groups borne by at least one of compounds X and Y is/are able to establish a hydrogen bond, and include either a hydrogen atom bound to an electronegative atom, or an electronegative atom for example an oxygen, nitrogen or sulphur atom. When the group has a hydrogen atom bound to an electronegative atom, the hydrogen atom can interact with another electronegative atom borne for example by another molecule, such as keratin, to form a hydrogen bond. When the group has an electronegative atom, the electronegative atom can interact with a hydrogen atom bound to an electronegative atom borne for example by another molecule, such as keratin, to form a hydrogen bond.

Advantageously, these polar groups can be selected from the following groups:
carboxylic acids —COOH,
alcohols, such as: —CH₂OH or —CH(R)OH, R being an alkyl radical having from 1 to 6 carbon atoms,
amino of formula —NR₁R₂, in which R₁ and R₂, which may be identical or different, represent an alkyl radical having from 1 to 6 carbon atoms or one of R₁ or R₂ denotes a hydrogen atom, and the other one of R₁ and R₂ represents an alkyl radical having from 1 to 6 carbon atoms,
pyridino,
amido of formula —NH—COR′ or —CO—NH—R′ in which R′ represents a hydrogen atom or an alkyl radical having from 1 to 6 carbon atoms,
pyrrolidino preferably selected from the groups of formula:

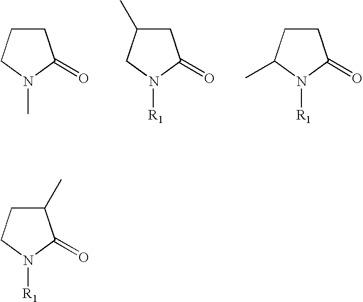

R₁ being an alkyl radical having from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulphonamido such as —NR′—S(═O)₂—R′, R′ corresponding to the above definition.

Preferably, these polar groups are present at a content less than or equal to 10 wt. % relative to the weight of each compound X or Y, preferably less than or equal to 5 wt. %, for example at a content ranging from 1 to 3 wt. %.

The polar group or groups can be located in the main chain of compound X and/or Y or can be pendant from the main chain or located at the ends of the main chain of compound X and/or Y.

1—Compounds X and Y Capable of Reacting by Hydrosilylation

According to one embodiment, the invention relates to a cosmetic composition intended for the care and/or make-up of keratinous substance(s) comprising, in a physiologically acceptable medium, at least one compound X, one compound Y, and at least one catalyst, with at least one of the compounds X or Y being a polyorganosiloxane, and said compounds X and Y being capable of reacting together by a hydrosilylation reaction in the presence of a catalyst, with at least one compound from the compounds X and Y being present in said composition in an encapsulated form, said catalyst being combined with at least one of the encapsulated compounds X or Y.

According to this embodiment, compounds X and Y are capable of reacting by hydrosilylation in the presence of a catalyst, said reaction being represented schematically in a simplified manner as follows:

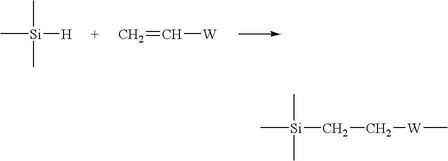

with W representing a carbon chain and/or silicone chain containing one or more unsaturated aliphatic groups.

In this case, compound X can be selected from silicone compounds comprising at least two unsaturated aliphatic groups. As an example, compound X can be a polyorganosiloxane comprising a silicone main chain whose unsaturated aliphatic groups are pendant from the main chain (side group) or located at the ends of the main chain of the compound (end group). These particular compounds will be called, hereinafter, polyorganosiloxanes with unsaturated aliphatic groups.

According to one embodiment, compound X and/or compound Y bear at least one polar group, as described above, capable of forming at least one hydrogen bond with keratinous substances. This polar group is advantageously carried by compound X, which has at least two unsaturated aliphatic groups.

According to one embodiment, compound X is selected from the polyorganosiloxanes comprising at least two unsaturated aliphatic groups, for example two or three vinyl or allyl groups, each attached to a silicon atom.

According to an advantageous embodiment, compound X is selected from the polyorganosiloxanes containing siloxane units of formula:

$${R}_{m} {R}^{′} {SiO}_{\frac{( 3 - m )}{2}} \qquad ( I )$$

in which:
R represents a linear or cyclic, monovalent hydrocarbon group, having from 1 to 30 carbon atoms, preferably from 1 to 20, and better still from 1 to 10 carbon atoms, for example a short-chain alkyl radical, comprising for example from 1 to 10 carbon atoms, in particular a methyl radical or alternatively a phenyl group, preferably a methyl radical,
m is equal to 1 or 2 and
R′ represents:
   an unsaturated aliphatic hydrocarbon group having from 2 to 10, preferably from 3 to 5 carbon atoms, for example a vinyl group or a group —R″-CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom; we may mention, as group R′, the vinyl and allyl groups and mixtures thereof; or
   an unsaturated cyclic hydrocarbon group having from 5 to 8 carbon atoms, for example a cyclohexenyl group.

Preferably R′ is an unsaturated aliphatic hydrocarbon group, preferably a vinyl group.

According to one embodiment, R represents an alkyl radical having from 1 to 10 carbon atoms or alternatively a phenyl group, and preferably a methyl radical, and R′ is a vinyl group.

According to a particular embodiment, the polyorganosiloxane also contains units of formula:

$${R}_{n} {SiO}_{\frac{( 4 - n )}{2}} \qquad ( II )$$

in which R is a group as defined previously, and n is equal to 1, 2 or 3.

According to a variant, compound X can be a silicone resin comprising at least two ethylenic unsaturations, said resin being capable of reacting with compound Y by hydrosilylation in the presence of a catalyst. We may mention for example the resins of type MQ or MT which themselves bear —CH═CH₂ unsaturated reactive end groups.

These resins are crosslinked organosiloxane polymers.

The class of the silicone resins is known by the name “MDTQ”, the resin being described in relation to the different siloxane monomer units that it contains, each of the letters “MDTQ” characterizing a type of unit.

The letter M represents the monofunctional unit of formula (CH₃)₃SiO_{1/2}, the silicon atom being bound to a single oxygen atom in the polymer comprising said unit.

The letter D denotes a bifunctional unit (CH₃)₂SiO_{2/2} in which the silicon atom is bound to two oxygen atoms.

The letter T represents a trifunctional unit of formula (CH₃)SiO_{3/2}.

In units M, D, T defined above, at least one of the methyl groups can be substituted with a group R other than the methyl group, such as a hydrocarbon radical (notably alkyl) having from 2 to 10 carbon atoms or a phenyl group or alternatively a hydroxyl group.

Finally, the letter Q denotes a tetrafunctional unit SiO_{4/2} in which the silicon atom is bound to four hydrogen atoms which are themselves attached to the rest of the polymer. As examples of said resins, we may mention the MT silicone resins such as poly(phenyl-vinylsilsesquioxane) such as that marketed under the reference SST-3PV1 by the company Gelest.

Preferably, compounds X have from 0.01 to 1 wt. % of unsaturated aliphatic groups.

Advantageously, compound X is selected from the polyorganopolysiloxanes, notably those comprising the siloxane units (I) and optionally (II) described previously.

Compound Y preferably has at least two free Si—H groups (hydrogenosilane groups).

Compound Y can be selected advantageously from the polyorganosiloxanes comprising at least one alkylhydrogenosiloxane unit of the following formula:

$${R}_{p} {HSiO}_{\frac{( 3 - p )}{2}} \qquad ( III )$$

in which:

R represents a linear or cyclic, monovalent hydrocarbon group, having from 1 to 30 carbon atoms, for example an alkyl radical having from 1 to 30 carbon atoms, preferably from 1 to 20 and better still from 1 to 10 carbon atoms, in particular a methyl radical, or alternatively a phenyl group and p is equal to 1 or 2. Preferably R is a hydrocarbon group, preferably methyl.

These polyorganosiloxane compounds Y with alkylhydrogenosiloxane units can additionally contain units of formula:

$${R}_{n} {SiO}_{\frac{( 4 - n )}{2}} \qquad ( II )$$

as defined above.

Compound Y can be a silicone resin comprising at least one unit selected from the units M, D, T, Q as defined above and comprising at least one Si—H group such as the poly(methyl-hydridosilsesquioxane) marketed under the reference SST-3 MH1.1 by the company Gelest.

Preferably, these polyorganosiloxane compounds Y have from 0.5 to 2.5 wt. % of Si—H groups.

Advantageously, the radicals R represent a methyl group in formulae (I), (II), (III) above.

Preferably, these polyorganosiloxanes Y have end groups of formula (CH₃)₃SiO_{1/2}.

Advantageously, the polyorganosiloxanes Y have at least two alkylhydrogenosiloxane units of formula —(H₃C)(H)SiO— and optionally include —(H₃C)₂SiO— units.

These polyorganosiloxane compounds Y with hydrogenosilane groups are described for example in document EP 0465744.

According to one variant, compound X is selected from the organic oligomers or polymers (by organic, we mean compounds whose main chain is not a silicone chain, preferably compounds not containing silicon atoms) or from hybrid organic/silicone polymers or oligomers, said oligomers or polymers bearing at least 2 unsaturated reactive aliphatic groups, compound Y being selected from the polyorganosiloxanes Y with hydrogenosilane groups mentioned above.

According to one embodiment, the organic or hybrid organic/silicone compounds X bearing at least 2 unsaturated reactive aliphatic groups, have at least one polar group as described above.

Compound X, of organic nature, can then be selected from the vinylic, (meth)acrylic polymers or oligomers, polyesters, polyurethanes and/or polyureas, polyethers, perfluoropolyethers, polyolefins such as polybutene, polyisobutylene, dendrimers or organic hyperbranched polymers, or mixtures thereof.

In particular, the organic polymer or the organic moiety of the hybrid polymer can be selected from the following polymers:

a) polyesters with ethylenic unsaturation(s):

This is a group of polymers of the polyester type having at least 2 ethylenic double bonds, randomly distributed in the main chain of the polymer. These unsaturated polyesters are obtained by polycondensation of a mixture:
of linear or branched aliphatic or cycloaliphatic dicarboxylic acids notably having 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with ethylenic unsaturations such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and/or of diol dimers resulting from reduction of dimers of fatty acids as defined previously, and
of one or more dicarboxylic acids or their anhydrides having at least one polymerizable ethylenic double bond and having from 3 to 50 carbon atoms, preferably from 3 to 20 and better still from 3 to 10 carbon atoms, such as maleic acid, fumaric acid or itaconic acid.

b) polyesters with (meth)acrylate side and/or end groups:

This is a group of polymers of the polyester type obtained by polycondensation of a mixture:
of linear or branched aliphatic or cycloaliphatic dicarboxylic acids notably having from 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with an ethylenic unsaturation such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyesters differ from those described above in section a) by the fact that the ethylenic double bonds are not located in the main chain but on side groups or at the end of the chains. These ethylenic double bonds are those of the (meth)acrylate groups present in the polymer.

Such polyesters are marketed for example by the company UCB under the designations EBECRYL® (EBECRYL® 450: molecular weight 1600, on average 6 acrylate functions per molecule, EBECRYL® 652: molecular weight 1500, on average 6 acrylate functions per molecule, EBECRYL® 800: molecular weight 780, on average 4 acrylate functions per molecule, EBECRYL® 810: molecular weight 1000, on average 4 acrylate functions per molecule, EBECRYL® 50 000: molecular weight 1500, on average 6 acrylate functions per molecule).

c) polyurethanes and/or polyureas with (meth)acrylate groups, obtained by polycondensation:
of aliphatic, cycloaliphatic and/or aromatic diisocyanates, triisocyanates and/or polyisocyanates notably having from 4 to 50, preferably from 4 to 30 carbon atoms, such as hexamethylenediisocyanate, isophoronediisocyanate, toluenediisocyanate, diphenylmethanediisocyanate or isocyanurates of formula:

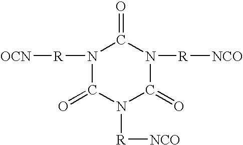

resulting from the trimerization of 3 molecules of diisocyanates OCN—R—CNO, where R is a linear, branched or cyclic hydrocarbon radical having from 2 to 30 carbon atoms;
of polyols, notably of diols, free from polymerizable ethylenic unsaturations, such as 1,4-butanediol, ethylene glycol or trimethylolpropane, and/or of polyamines, notably of aliphatic, cycloaliphatic and/or aromatic diamines, notably having from 3 to 50 carbon atoms, such as ethylenediamine or hexamethylenediamine, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyurethanes/polyureas with acrylate groups are marketed for example under the designation SR 368 (tris(2-hydroxyethyl)isocyanurate-triacrylate) or CRAYNOR® 435 by the company CRAY VALLEY, or under the designation EBECRYL® by the company UCB (EBECRYL® 210: molecular weight 1500, 2 acrylate functions per molecule, EBECRYL® 230: molecular weight 5000, 2 acrylate functions per molecule, EBECRYL®& 270: molecular weight 1500, 2 acrylate functions per molecule, EBECRYL® 8402: molecular weight 1000, 2 acrylate functions per molecule, EBECRYL® 8804: molecular weight 1300, 2 acrylate functions per molecule, EBECRYL® 220: molecular weight 1000, 6 acrylate functions per molecule, EBECRYL® 2220: molecular weight 1200, 6 acrylate functions per molecule, EBECRYL® 1290: molecular weight 1000, 6 acrylate functions per molecule, EBECRYL® 800: molecular weight 800, 6 acrylate functions per molecule).

We may also mention the water-soluble aliphatic diacrylate polyurethanes marketed under the designations EBECRYL® 2000, EBECRYL® 2001 and EBECRYL® 2002, and the diacrylate polyurethanes in aqueous dispersion marketed under the trade names IRR® 390, IRR® 400, IRR® 422 IRR® 424 by the company UCB.

d) polyethers with (meth)acrylate groups obtained by esterification, by (meth)acrylic acid, of the hydroxyl end groups of homopolymers or of C₁₋₄ alkylene glycol copolymers, such as polyethylene glycol, polypropylene glycol, copolymers of ethylene oxide and of propylene oxide preferably having a weight-average molecular weight below 10 000, polyethoxylated or polypropoxylated trimethylolpropane.

Di(meth)acrylate polyoxyethylenes of suitable molecular weight are marketed for example under the designations SR 259, SR 344, SR 610, SR 210, SR 603 and SR 252 by the company CRAY VALLEY or under the designation EBECRYL® 11 by UCB. Polyethoxylated trimethylolpropane triacrylates are marketed for example under the designations SR 454, SR 498, SR 502, SR 9035, SR 415 by the company CRAY VALLEY or under the designation EBECRYL® 160 by the company UCB. Polypropoxylated trimethylolpropane triacrylates are marketed for example under the designations SR 492 and SR 501 by the company CRAY VALLEY.

e) epoxyacrylates obtained by reaction between
at least one diepoxide selected for example from:
   (i) bisphenol A diglycidyl ether,
   (ii) a diepoxy resin resulting from the reaction between bisphenol A diglycidyl ether and epichlorohydrin,
   (iii) an epoxyester resin with α,ω-diepoxy end groups resulting from the condensation of a dicarboxylic acid having from 3 to 50 carbon atoms with a stoichiometric excess of (i) and/or (ii),
   (iv) an epoxyether resin with α,ω-diepoxy end groups resulting from the condensation of a diol having from 3 to 50 carbon atoms with a stoichiometric excess of (i) and/or (ii),
   (v) natural or synthetic oils bearing at least 2 epoxide groups, such as epoxidized soya oil, epoxidized linseed oil and epoxidized vernonia oil,
   (vi) a phenol-formaldehyde polycondensate (Novolac® resin), of which the end groups and/or side groups have been epoxidized, and
one or more carboxylic acids or carboxylic polyacids having at least one ethylenic double bond at α,β of the carboxyl group such as (meth)acrylic acid or crotonic acid or esters of (meth)acrylic acid and of a diol or polyol having from 2 to 20 carbon atoms, preferably from 2 to 6 carbon atoms such as 2-hydroxyethyl (meth)acrylate.

Such polymers are marketed for example under the designations SR 349, SR 601, CD 541, SR 602, SR 9036, SR 348, CD 540, SR 480, CD 9038 by the company CRAY VALLEY, under the designations EBECRYL® 600 and EBECRYL® 609, EBECRYL® 150, EBECRYL® 860, EBECRYL® 3702 by the company UCB and under the designations PHOTOMER® 3005 and PHOTOMER® 3082 by the company HENKEL.

f) (C₁₋₅₀ alkyl) poly(meth)acrylates, said alkyl being linear, branched or cyclic, bearing at least two functions with ethylenic double bond carried by the lateral and/or terminal hydrocarbon chains.

Such copolymers are marketed for example under the designations IRR® 375, OTA® 480 and EBECRYL® 2047 by the company UCB.

g) polyolefins such as polybutene, polyisobutylene,

h) perfluoropolyethers with acrylate groups obtained by esterification, for example by (meth)acrylic acid, of perfluoropolyethers bearing hydroxyl side and/or end groups.

These α,ω-diol perfluoropolyethers are described notably in EP-A-1057849 and are marketed by the company AUSIMONT under the designation FOMBLIN® Z DIOL.

i) dendrimers and hyperbranched polymers bearing (meth)acrylate or (meth)acrylamide end groups obtained respectively by esterification or amidation of dendrimers and of hyperbranched polymers with hydroxyl or amino terminal functions, by (meth)acrylic acid.

The dendrimers (from the Greek dendron=tree) are “tree-like” polymer molecules, i.e. highly branched, invented by D. A. Tomalia and his team at the beginning of the 1990's (Donald A. Tomalia et al., Angewandte Chemie, Int. Engl. Ed., Vol. 29, No. 2, pages 138-175). They are structures constructed around a, generally polyvalent, central unit. Branched chain-extending units are arranged according to a perfectly defined structure around this central unit, thus giving rise to symmetrical, monodispersed macromolecules having a well-defined chemical and stereochemical structure. Dendrimers of the polyamidoamine type are marketed for example under the name STARBURST® by the company DENDRITECH.

The hyperbranched polymers are polycondensates, generally of the polyester, polyamide or polyethyleneamine type, obtained from multifunctional monomers, which have a tree-like structure similar to that of the dendrimers but far less regular than the latter (see for example WO-A-93/17060 and WO 96/12754).

The company PERSTORP markets hyperbranched polyesters under the name BOLTORN®. Hyperbranched polyethyleneamines are available under the name COMBURST® from the company DENDRITECH. Hyperbranched poly(esteramide)s with hydroxyl end groups are marketed by the company DSM under the name HYBRANE®.

These dendrimers and hyperbranched polymers, esterified or amidated by acrylic and/or methacrylic acid, differ from the polymers described in sections a) to h) above by the very large number of ethylenic double bonds present. This increased functionality, generally greater than 5, makes them particularly useful in enabling them to act as a “crosslinking node”, i.e. a multiple crosslinking site.

It is therefore possible to use these dendritic and hyperbranched polymers in association with one or more of the above polymers and/or oligomers a) to h).

1a—Additional Reactive Compounds

According to one embodiment, the compositions containing compound X and/or Y can additionally comprise an additional reactive compound such as:
organic or mineral particles having on their surface at least 2 unsaturated aliphatic groups—we may mention for example the silicas surface-treated for example with silicone compounds with vinylic groups such as for example cyclotetramethyltetravinylsiloxane-treated silica,
silazane compounds such as hexamethyldisilazane.

1b—Catalyst

The hydrosilylation reaction takes place in the presence of a catalyst which can be present with one or other of the compounds X or Y or can be present on its own. For example, this catalyst can be present in the composition in an encapsulated form if the two compounds X and Y, which it must cause to interact, are present in this same composition in an unencapsulated form or conversely it can be contained there in an unencapsulated form if at least one of compounds X and Y is present in the composition in an encapsulated form. The catalyst is preferably based on platinum or tin.

We may mention for example platinum-based catalysts deposited on a support of silica gel or of powdered charcoal, platinum chloride, salts of platinum and of chloroplatinic acids.

The chloroplatinic acids are preferably used in hexahydrate or anhydrous form, which are easily dispersible in organosilicone media.

We may also mention platinum complexes, such as those based on chloroplatinic acid hexahydrate and divinyl tetramethyldisiloxane.

The catalyst can be present at a content in the range from 0.0001 to 20 wt. % relative to the total weight of the composition containing it.

Compounds X and/or Y can be combined with polymerization inhibitors or retarders, and more particularly inhibitors of the catalyst. Non-limitatively, we may mention cyclic polymethylvinylsiloxanes, and in particular tetravinyl tetramethyl cyclotetrasiloxane, acetylenic alcohols, preferably volatile, such as methylisobutynol.

The presence of ionic salts, such as sodium acetate, can have an influence on the rate of polymerization of the compounds.

As an example of a combination of compounds X and Y reacting by hydrosilylation in the presence of a catalyst, we may mention the following references offered by the company Dow Corning: DC7-9800 Soft Skin Adhesive Parts A & B, as well as the combination of the following mixtures A and B prepared by Dow Corning:

Mixture A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

Mixture B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

Advantageously, compounds X and Y are selected from silicone compounds capable of reacting by hydrosilylation in the presence of a catalyst; in particular compound X is selected from the polyorganosiloxanes comprising units of formula (I) described above and compound Y is selected from organosiloxanes comprising alkylhydrogenosiloxane units of formula (III) described above.

According to a particular embodiment, compound X is a polydimethylsiloxane with vinylic end groups, and compound Y is a polymethylhydrogenosiloxane.

According to one particular embodiment, the catalyst is used in an encapsulated form with a compound X such as defined previously, and especially is encapsulated with a compound X chosen from the polyorganosiloxanes comprising at least two unsaturated aliphatic groups.

2/Compounds X and Y Capable of Reacting by Condensation

According to one embodiment, the invention relates to a cosmetic composition intended for the care and/or make-up of keratinous substance(s) comprising, in a physiologically acceptable medium, at least one compound X and one compound Y, and optionally a catalyst, with at least one of compounds X and Y being a polyorganosiloxane, and said compounds X and Y being capable of reacting together by a condensation reaction, with at least one compound from the compounds X, Y and the catalyst, when it is present, being present in said composition in an encapsulated form.

According to this embodiment, compounds X and Y are capable of reacting by condensation, either in the presence of water (hydrolysis) by reaction of 2 compounds bearing alkoxysilane groups, or by so-called “direct” condensation by reaction of a compound bearing alkoxysilane group(s) and a compound bearing silanol group(s) or by reaction of 2 compounds bearing silanol group(s).

When the condensation is carried out in the presence of water, the latter can in particular be the ambient humidity, the residual water of the skin, of the lips, of the eyelashes and/or of the nails, or water from an external source, for example by prior moistening of the keratinous substance (for example by an atomizer, by natural or artificial tears).

In this manner of reaction by condensation, compounds X and Y, which may be identical or different, can therefore be selected from silicone compounds whose main chain contains at least two alkoxysilane groups and/or at least two silanol (Si—OH) side groups or end groups.

According to one embodiment, compound X and/or compound Y bears at least one polar group, as described above, capable of forming at least one hydrogen bond with keratinous substances.

According to an advantageous embodiment, compounds X and/or Y are selected from the polyorganosiloxanes comprising at least two alkoxysilane groups. By “alkoxysilane group”, we mean a group comprising at least one —Si—OR moiety, R being an alkyl group having from 1 to 6 carbon atoms.

Compounds X and Y are notably selected from the polyorganosiloxanes comprising alkoxysilane end groups, more specifically those which have at least 2 alkoxysilane end groups, preferably trialkoxysilane end groups.

These compounds X and/or Y preferably mostly comprise units of formula:

R⁹ₛSiO_{(4-s)/2}, (IV)

in which the groups R⁹ represent, independently of one another, a radical selected from alkyl groups having from 1 to 6 carbon atoms, phenyl groups, fluoroalkyl groups, and s is equal to 0, 1, 2 or 3. Preferably, groups R⁹ represent, independently of one another, an alkyl group having from 1 to 6 carbon atoms. As alkyl group, we may notably mention methyl, propyl, butyl, hexyl and mixtures thereof, preferably methyl or ethyl. As fluoroalkyl group, we may mention 3,3,3-trifluoropropyl.

According to a particular embodiment, compounds X and Y, which may be identical or different, are polyorganosiloxanes comprising units of formula:

(R⁹₂SiO₂)_{f}— (V)

in which R⁹ is as described above, preferably R⁹ is a methyl radical, and f is such that the polymer advantageously has a viscosity at 25° C. in the range from 0.5 to 3000 Pa·s, preferably in the range from 5 to 150 Pa·s; for example f can range from 2 to 5000, preferably from 3 to 3000, and more preferably from 5 to 1000.

These polyorganosiloxane compounds X and Y contain at least 2 trialkoxysilane end groups per molecule of polymer, said groups having the following formula

-ZSiR¹ₓ(OR)₃₋ₓ, (VI)

in which:

the radicals R represent, independently, a methyl, ethyl, n-propyl, isopropyl, n-butyl, sec-butyl, isobutyl group, preferably a methyl or ethyl group,

R¹ is a methyl or ethyl group,

x is equal to 0 or 1, preferably x is equal to 0 and

Z is selected from: the divalent hydrocarbon groups that do not have an ethylenic unsaturation and have from 1 to 18 carbon atoms, preferably from 2 to 18 carbon atoms (alkylene groups), the combinations of divalent hydrocarbon radicals and siloxane segments of the following formula (IX):

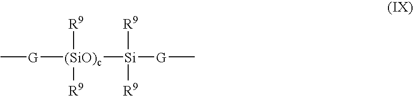

R⁹ being as described above, G is a divalent hydrocarbon radical without an ethylenic unsaturation and having from 1 to 18 carbon atoms, preferably from 2 to 18 carbon atoms and c is an integer in the range from 1 to 6.

Z and G can notably be selected from the alkylene groups such as methylene, ethylene, propylene, butylene, pentylene, hexylene, the arylene groups such as phenylene.

Preferably, Z is an alkylene group, and more preferably ethylene.

These polymers can have on average at least 1.2 trialkoxysilane end groups or terminal chains per molecule, and preferably on average at least 1.5 trialkoxysilane end groups per molecule. These polymers that can have at least 1.2 trialkoxysilane end groups per molecule, some can include other types of end groups such as end groups of formula CH₂═CH—SiR⁹₂— or of formula R⁶₃—Si—, in which R⁹ is as defined previously and each group R⁶ is selected independently from the R⁹ or vinyl groups. As examples of said end groups, we may mention the trimethoxysilane, triethoxysilane, vinyldimethoxysilane and vinylmethyloxyphenylsilane groups.

Such polymers are notably described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the contents of which are incorporated by reference in the present application.

We may mention, as compound X and/or Y, in particular the polyorganosiloxanes selected from the polymers of formula:

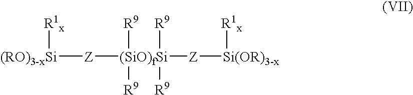

in which R, R¹, R⁹, Z, x and f are as described above.

Compounds X and/or Y can also include a mixture of polymers of formula (VII) above with polymers of the following formula (VIII):

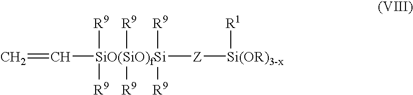

in which R, R¹, R⁹, Z, x, and f are as described above.

When the polyorganosiloxane compound X and/or Y with alkoxysilane group(s) includes said mixture, the various polyorganosiloxanes are present at contents such that the organosilyl terminal chains represent less than 40%, preferably less than 25% in number of terminal chains.

Polyorganosiloxane compounds X and/or Y that are particularly preferred are those of formula (VII) described above. Such compounds X and/or Y are described for example in document WO 01/96450.

As stated above, compounds X and Y can be identical or different.

In particular, compounds X and Y can represent a mixture of polydimethylsiloxanes with methoxysilane groups.

According to a variant, one of the 2 reacting compounds X or Y is of silicone character and the other is of organic character. For example, compound X is selected from organic oligomers or polymers or hybrid organic/silicone oligomers or polymers, said polymers or oligomers comprising at least two alkoxysilane groups, and Y is selected from silicone compounds such as the polyorganosiloxanes described above. In particular, the organic oligomers or polymers are selected from the vinylic, (meth)acrylic oligomers or polymers, polyesters, polyamides, polyurethanes and/or polyureas, polyethers, polyolefins, perfluoropolyethers, dendrimers and hyperbranched organic polymers, and mixtures thereof.

According to one embodiment, compound X of organic character or of hybrid organic/silicone character bears at least one polar group, as described above, capable of forming at least one hydrogen bond with the keratinous substance.

The organic polymers of vinylic or (meth)acrylic character, bearing alkoxysilane side groups, can in particular be obtained by copolymerization of at least one vinylic or (meth)acrylic organic monomer with a (meth)acryloxypropyltrimethoxysilane, a vinyltrimethoxysilane, a vinyltriethoxysilane, an allyltrimethoxysilane etc.

We may mention for example the (meth)acrylic polymers described in the document of KUSABE, M, Pitture e Vemiei—European Coating; 12-B, pages 43-49, 2005, and notably the polyacrylates with alkoxysilane groups with the designation MAX from Kaneka or those described in the work by PROBSTER, M, Adhesion-Kleben & Dichten, 2004, 481 (1-2), pages 12-14.

The organic polymers resulting from a polycondensation or a polyaddition, such as polyesters, polyamides, polyurethanes and/or polyureas, polyethers, and bearing alkoxysilane side and/or end groups, can result for example from reaction of an oligomeric prepolymer as described above with one of the following silane reaction partners bearing at least one alkoxysilane group: aminopropyltrimethoxysilane, aminopropyltriethoxysilane, aminoethyl aminopropyl trimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes with alkoxysilane groups are described in the work by KUSABE, M., Pitture e Vemiei—European Coating; 12-B, pages 43-49, 2005. As examples of polyurethanes with alkoxysilane end groups, we may mention those described in the document PROBSTER, M., Adhesion-Kleben & Dichten, 2004, 481 (1-2), pages 12-14 or alternatively those described in the document LANDON, S., Pitture e Verniei Vol. 73, No. 11, pages 18-24, 1997 or in the document HUANG, Mowo, Pitture e Verniei Vol. 5, 2000, pages 61-67, and we may notably mention the polyurethanes with alkoxysilane groups from OSI-WITCO-GE.

As polyorganosiloxane compounds X and/or Y, we may mention the resins of type MQ or MT which themselves bear alkoxysilane and/or silanol end groups, for example the poly(isobutylsilsesquioxane) resins functionalized with silanol groups offered under reference SST-S7C41 (three Si—OH groups) by the company Gelest.

2a —Additional Reactive Compound

According to one embodiment, compound X and/or Y can additionally be combined with an additional reactive compound comprising at least two alkoxysilane or silanol groups.

We may mention for example:
one or more organic or mineral particles with alkoxysilane and/or silanol groups on their surface, for example fillers surface-treated with said groups.

2b—Catalyst

The condensation reaction can take place in the presence of a metal-based catalyst which can be present with one or other of the compounds X or Y or can be present on its own. For example, said catalyst can be present in the composition in an encapsulated form if the two compounds X and Y, which it is to cause to interact, are present in this same composition in an unencapsulated form or conversely it can be present there in an unencapsulated form if at least one of compounds X and Y is present in the composition in an encapsulated form. The catalyst for use in this type of reaction is preferably a titanium-based catalyst.

We may notably mention the catalysts based on tetraalkoxytitanium of formula:

Ti(OR²)_{y}(OR³)_{4-y},

in which R² is selected from the tertiary alkyl radicals such as tert-butyl, tert-amyl and 2,4-dimethyl-3-pentyl; R³ represents an alkyl radical having from 1 to 6 carbon atoms, preferably a methyl, ethyl, n-propyl, isopropyl, n-butyl, sec-butyl, hexyl group and

y is a number in the range from 3 to 4, preferably from 3.4 to 4.

The catalyst can be present at a content ranging from 0.0001 to 20 wt. % relative to the total weight of the composition containing it.

2c—Diluent

The compositions that can be used, comprising X and/or Y, can additionally include a volatile silicone oil (or diluent) for lowering the viscosity of the composition. Said oil can be selected from the short-chain linear silicones such as hexamethyldisiloxane, octamethyltrisiloxane, cyclic silicones such as octamethylcyclotetrasiloxane, decamethylcyclopentasiloxane and mixtures thereof.

This silicone oil can represent from 5 to 95 wt. %, preferably from 10 to 80 wt. % relative to the weight of each composition.

As an example of a combination of compounds X and Y bearing alkoxysilane groups and reacting by condensation, we may mention the combination of the following mixtures A′ and B′ produced by the company Dow Corning:

Mixture A′:

| Ingredient (INCI name) | CAS No. | Contents (%) | Function |
|---|---|---|---|
| Bis- | PMN87176 | 25-45 | Polymer |
| Trimethoxysiloxyethyl | | | |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| Ingredient (INCI name) | CAS No. | Contents (%) | Function |
|---|---|---|---|
| Disiloxane | 107-46-0 | 80-99 | Solvent |
| Tetra T Butyl Titanate | — | 1-20 | Catalyst |

It should be noted that compounds X and Y, identical, are combined in mixture A′ (cf. (1)).

3/ Crosslinking in the Presence of Peroxide:

According to one embodiment, the invention relates to a cosmetic composition intended for the care and/or make-up of keratinous substance(s) comprising, in a physiologically acceptable medium, at least one compound X, one compound Y, and a peroxide, with at least one of compounds X and Y being a polyorganosiloxane, and said compounds X and Y being capable of reacting together by a crosslinking reaction in the presence of a peroxide, with at least one compound from the compounds X, Y and the peroxide being present in said composition in an encapsulated form.

This reaction is preferably effected by heating to a temperature greater than or equal to 50° C., preferably greater than or equal to 80° C., and up to 120° C.

Compounds X and Y, which may be identical or different, have in this case at least two —CH₃ side groups and/or at least two side chains bearing a —CH₃ group.

Compounds X and Y are preferably silicone compounds and can be selected for example from the non-volatile linear polydimethylsiloxanes of high molecular weight, having a degree of polymerization above 6 and with at least two —CH₃ side groups attached to the silicon atom and/or at least two side chains bearing a —CH₃ group. We may mention for example the polymers described in the Catalogue “Reactive Silicones” of the company Gelest Inc., Edition 2004, page 6, and notably the copolymers (also called gums) of vinylmethylsiloxane-dimethylsiloxane of molecular weight in the range from 500 000 to 900 000 and notably with viscosity above 2 000 000 cSt.

As peroxides that can be used in the invention, we may mention benzoyl peroxide, 2,4-dichlorobenzoyl peroxide and mixtures thereof.

According to one embodiment, the hydrosilylation reaction in the presence of a catalyst, or the condensation reaction, or alternatively the crosslinking reaction in the presence of a peroxide, between compounds X and Y is accelerated by supply of heat, for example by raising the temperature of the system between 25° C. and 180° C.

In general, regardless of the type of reaction by which compounds X and Y react with one another, the molar percentage of X relative to the total of compounds X and Y, i.e. the ratio X/(X+Y)×100, can vary from 5 to 95%, preferably from 10 to 90%, and more preferably from 20 to 80%.

Similarly, the molar percentage of Y relative to the total of compounds X and Y, i.e. the ratio Y/(X+Y)×100, can vary from 5 to 95%, preferably from 10 to 90%, and more preferably from 20 to 80%.

Compound X can have a weight-average molecular weight (Mw) in the range from 150 to 1 000 000, preferably from 200 to 800 000, more preferably from 200 to 250 000.

Compound Y can have a weight-average molecular weight (Mw) in the range from 200 to 1 000 000, preferably from 300 to 800 000, more preferably from 500 to 250 000.

Compound X can represent from 0.1 to 95 wt. % relative to the total weight of the composition containing it, preferably from 1 to 90%, and more preferably from 5 to 80%.

Compound Y can represent from 0.1 to 95 wt. % relative to the total weight of the composition containing it, preferably from 1 to 90%, and more preferably from 5 to 80%.

The ratio of compound X to compound Y can be varied so as to adjust the reaction rate and therefore the rate of formation of the film or alternatively so as to adapt the properties of the film formed (for example its adhesive properties) according to the intended application.

In particular, compounds X and Y can be present at a molar ratio X/Y in the range from 0.05 to 20 and preferably from 0.1 to 10.

Compounds X and Y can advantageously be combined with at least one filler. Thus, the kit according to the invention can for example include, in at least one of the compositions, a filler selected from silica or surface-treated silica.

Physiologically Acceptable Medium

As specified previously, a composition according to the invention comprises a physiologically acceptable medium.

The expression “physiologically acceptable medium” is understood to mean a medium that is particularly suitable for the application of a composition according to the invention on a keratinous substance such as the skin or the lips. The physiologically acceptable medium is generally adapted to the nature of the support on which the composition must be applied, and also to the form in which the composition is intended to be packaged.

The physiologically acceptable medium may comprise an aqueous phase, possibly mainly consisting of water.

It may also comprise a mixture of water and a solvent that is miscible with water (miscibility in water greater than 50 wt % at 25° C.) such as a, or a mixture of lower monoalcohol(s) having from 1 to 5 carbon atoms such as ethanol, isopropanol, glycols having from 2 to 8 carbon atoms such as propylene glycol, ethylene glycol, 1,3-butylene glycol, dipropylene glycol, C₃-C₄ ketones, C₂-C₄ aldehydes and mixtures thereof.

The aqueous phase (water and optionally the solvent that is miscible with water) may be present in an amount ranging from 5 to 95% by weight relative to the total weight of the composition, preferably from 10% to 85% by weight and better still from 2 to 80% by weight.

The physiologically acceptable medium may also comprise a liquid fatty phase, comprising one or more volatile or non-volatile oils, and/or a solid fatty phase comprising one or more waxes and/or pasty compounds, and a mixture thereof.

The expression “liquid fatty phase”, in the sense of the Application, is understood to mean a fatty phase that is liquid at ambient temperature (25° C.) and atmospheric pressure (760 mmHg), composed of one or more non-aqueous fatty substances that are liquid at ambient temperature, also known as oils or organic solvents.

The oil may be chosen from volatile oils and/or non-volatile oils, and mixtures thereof.

The oil or oils may be present in an amount ranging from 1% to 90% by weight, preferably from 5% to 50% by weight relative to the total weight of the composition.

These oils may be hydrocarbon-based oils, silicone oils, fluorinated oils or mixtures thereof.

The expression “hydrocarbon-based oil” is understood to mean an oil that mainly contains hydrogen and carbon atoms and optionally oxygen, nitrogen, sulphur or phosphorus atoms. The hydrocarbon-based oils may be chosen from hydrocarbon-based oils having from 8 to 16 carbon atoms, and especially C₈-C₁₆ branched alkanes such as C₈-C₁₆ isoalkanes of petroleum origin (also known as isoparaffins) such as isododecane (also known as 2,2,4,4,6-pentamethylheptane), isodecane, isohexadecane, and for example the oils sold under the commercial names ISOPARS® or PERMETYLS®, C₈-C₁₆ branched esters, isohexyl neopentanoate, and mixtures thereof.

As hydrocarbon-based oil, mention may also be made of:

hydrocarbon-based oils of plant origin such as triesters of fatty acids and of glycerol, the fatty acids of which may have varied chain lengths from C₄ to C₂₄, the latter possibly being linear or branched, saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, marrow oil, sesame seed oil, pumpkin oil, rapeseed oil, blackcurrant oil, evening primrose oil, millet oil, barley oil, quinoa oil, rye oil, safflower oil, candlenut oil, passion flower oil, musk rose oil; or else caprylic/capric acid triglycerides such as those sold by Stearineries Dubois or those sold under the names MIGLYOL 810, 812 and 818 by Dynamit Nobel;

synthetic ethers having from 10 to 40 carbon atoms;

apolar hydrocarbon-based oils such as squalene, linear or branched hydrocarbons such as paraffin oil, vaseline oil and naphthalene oil, hydrogenated or partially hydrogenated polyisobutene, isoeicosane, squalane, decene/butene copolymers, polybutene/polyisobutene copolymers especially INDOPOL L-14, polydecenes such as PURESYN 10, and mixtures thereof;

synthetic esters such as oils of formula R₁COOR₂ in which R₁ represents the residue of a linear or branched fatty acid having from 1 to 40 carbon atoms and R₂ represents an in particular branched hydrocarbon-based chain containing from 1 to 40 carbon atoms on condition that R₁+R₂≧10, such as for example Purcellin oil (cetostearyl octanoate), isopropyl myristate, isopropyl palmitate, C₁₂ to C₁₅ alcohol benzoate, hexyl laurate, diisopropyl adipate, isononyl isononanoate, 2-ethylhexyl palmitate, isostearyl isostearate, alcohol or polyalcohol octanoates, decanoates or ricinoleates such as propylene glycol dioctanoate; hydroxylated esters such as isostearyl lactate, diisostearyl malate; and pentaerythritol esters;

fatty alcohols that are liquid at ambient temperature having a branched and/or unsaturated carbon-based chain having from 12 to 26 carbon atoms such as octyldodecanol, isostearyl alcohol, oleyl alcohol, 2-hexyldecanol, 2-butyloctanol, 2-undecylpentadecanol;

higher fatty acids such as oleic acid, linoleic acid, linolenic acid; and

mixtures thereof.

As a silicone oil that can be used in the invention, mention may especially be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

The silicone oils may also be:
polydimethylsiloxanes (PDMs);
polydimethylsiloxanes having alkyl or alkoxy groups, which are pendant and/or at the end of the silicone chain, groups that each have from 3 to 40 carbon atoms;
phenyl silicones such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethylsiloxysilicates; and
mixtures thereof.

The compositions according to the invention may also comprise at least one fatty substance that is solid at ambient temperature especially chosen from waxes, pasty fatty substances and mixtures thereof. These fatty substances may be of animal, plant, mineral or synthetic origin.

The compositions according to the invention may also contain ingredients commonly used in cosmetics, such as vitamins, thickeners, gelling agents, trace elements, softeners, sequestrants, fragrances, acidifying or basifying agents, preservatives, fragrances, sunscreens, surfactants, antioxidants, fibres, fillers, dyes, film-forming polymers, waxes, cosmetic active agents such as bactericidal or antiperspirant active agents, neutralizers, emollients, moisturizers, and mixtures thereof.

The compositions according to the invention may contain emulsifying surfactants present, in particular, in an amount ranging from 0.1 to 30% by weight relative to the total weight of the composition, better from 1 to 15% and better still from 2 to 10%.

These surfactants may be chosen from anionic, nonionic, amphoteric or zwitterionic surfactants. Reference may be made to the document “Encyclopedia of Chemical Technology, KIRK-OTHMER”, volume 22, p. 333-432, 3rd Edition, 1979, WILEY, for the definition of the (emulsifying) functions and properties of surfactants, in particular p. 347-377 of this reference, for anionic and nonionic surfactants.

The nonionic surfactants of the invention are preferably chosen from:

1—silicone surfactants;

2—surfactants that are liquid at a temperature of less than or equal to 45° C., chosen from the esters of at least one polyol of at least one fatty acid having at least one C₈-C₂₂ alkyl chain that is saturated or unsaturated, linear or branched, and especially unsaturated or branched, the polyol being chosen from the group formed by polyethylene glycol having 1 to 60 ethylene oxide units, sorbitan, glycerol which may have from 2 to 30 ethylene oxide units, polyglycerols having from 2 to 15 glycerol units;

3—fatty acid and sugar esters and fatty alcohol and sugar ethers;

4—surfactants that are solid at a temperature equal to 45° C., chosen from glycerol fatty esters, sorbitan fatty esters and oxyethylenated sorbitan fatty esters, ethoxylated fatty ethers and ethoxylated fatty esters;

5—block copolymers of ethylene oxide (A) and of propylene oxide (B); and mixtures of these surfactants.

1—The silicone surfactants that can be used according to the invention are silicone compounds having at least one oxyethylenated —OCH₂CH₂— and/or oxypropylenated —OCH₂CH₂CH₂— chain. As silicone surfactants which may be used according to the present invention, mention may be made of those described in documents U.S. Pat. No. 5,364,633 and U.S. Pat. No. 5,411,744.

Preferably, the silicone surfactant used according to the present invention is a compound of formula (II):

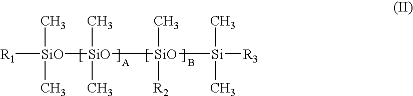

in which:

R₁, R₂, R₃, independently of one another, represent a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical; R₄ being a hydrogen, an alkyl radical or an acyl radical;

A is an integer ranging from 0 to 200;

B is an integer ranging from 0 to 50; on condition that A and B are not equal to zero at the same time;

x is an integer ranging from 1 to 6;

y is an integer ranging from 1 to 30; and

z is an integer ranging from 0 to 5.

According to one preferred embodiment of the invention, in the compound of formula (II), the alkyl radical is a methyl radical, x is an integer ranging from 2 to 6 and y is an integer ranging from 4 to 30.

Mention may be made, by way of example of silicone surfactants of formula (II), of compounds of formula (III):

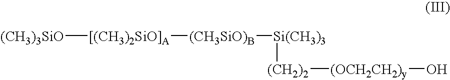

in which A is an integer ranging from 20 to 105, B is an integer ranging from 2 to 10 and y is an integer ranging from 10 to 20.

Mention may also be made, by way of example of silicone surfactants of formula (II), of compounds of formula (IV):

H—(OCH₂CH₂)_{y}—(CH₂)₃-[(CH₃)₂SiO]_{A′}—(CH₂)₃—(OCH₂CH₂)_{y}—OH (IV)

in which A′ and y are integers ranging from 10 to 20.

It is possible in particular to use, as silicone surfactants, those sold by Dow Corning under the names DC 5329, DC 7439-146, DC 2-5695 and Q4-3667. The compounds DC 5329, DC 7439-146, DC 2-5695 are compounds of formula (III) where A is 22, B is 2 and y is 12; A is 103, B is 10 and y is 12; A is 27, B is 3 and y is 12, respectively.

The compound Q4-3667 is a compound of formula (IV) where A is 15 and y is 13.

2—Surfactants that are liquid at a temperature less than or equal to 45° C. may especially be chosen from:
polyethylene glycol isostearate of molecular weight 400 (CTFA name: PEG-8 Isostearate), sold under the name PRISORINE 3644 by Unichema;
diglyceryl isostearate, sold by Solvay;
polyglycerol laurate having 2 glycerol units (polyglyceryl-2 laurate), sold under the name DIGLYCERIN-MONOLAURATE by Solvay;
sorbitan oleate, sold under the name SPAN 80 by ICI;
sorbitan isostearate, sold under the name NIKKOL SI 10R by Nikko; and
α-butylglycoside cocoate or α-butylglycoside caprate sold by Ulice.

3—The fatty acid and sugar esters that can be used as nonionic amphiphilic lipids in the nanoemulsion according to the invention are preferably solid at a temperature less than or equal to 45° C. and may be chosen, in particular, from the group comprising the esters or mixtures of esters of C₈-C₂₂ fatty acid and of sucrose, maltose, glucose or fructose, and the esters or mixtures of esters of C₁₄-C₂₂ fatty acid and of methylglucose.

The C₈-C₂₂ or C₁₄-C₂₂ fatty acids forming the fatty unit of the esters that can be used in the nanoemulsion of the invention have a saturated or unsaturated linear alkyl chain, respectively having from 8 to 22 or from 14 to 22 carbon atoms. The fatty unit of the esters may especially be chosen from stearates, behenates, arachidonates, palmitates, myristates, laurates, caprates and mixtures thereof. Preferably, stearates are used.

Mention may be made, by way of example of esters or mixtures of esters of fatty acid and of sucrose, maltose, glucose or fructose, of sucrose monostearate, sucrose distearate, sucrose tristearate and mixtures thereof, such as the products sold by Croda under the name CRODESTA F50, F70, F110 and F160 respectively having an HLB (Hydrophilic Lipophilic Balance) of 5, 7, 11 and 16; and by way of example of esters or mixtures of esters of fatty acid and of methylglucose, of the distearate of methylglucose and of polyglycerol-3, sold by Goldschmidt under the name TEGO-CARE 450. Mention may also be made of the monoesters of glucose or of maltose such as methyl o-hexadecanoyl-6-D-glucoside and methyl o-hexadecanoyl-6-D-maltoside.

The fatty alcohol and sugar ethers, that can be used as nonionic amphiphilic lipids in the nanoemulsion according to the invention are solid at a temperature less than or equal to 45° C. and may especially be chosen from the group comprising ethers or mixtures of ethers of C₈-C₂₂ fatty alcohol and of glucose, maltose, sucrose or fructose and the ethers or mixtures of ethers of C₁₄-C₂₂ fatty alcohol and of methylglucose. These are, in particular, alkyl polyglucosides.

The C₈-C₂₂ or C₁₄-C₂₂ fatty alcohols forming the fatty unit of the ethers that can be used in the nanoemulsion of the invention have a saturated or unsaturated linear alkyl chain, respectively having from 8 to 22 or from 14 to 22 carbon atoms. The fatty unit of the ethers may especially be chosen from decyl, cetyl, behenyl, arachidyl, stearyl, palmityl, myristyl, lauryl, capryl, hexadecanoyl units and mixtures thereof such as the cetearyl unit.

By way of example of fatty alcohol and sugar ethers, mention may be made of the alkyl polyglucosides such as decyl glucoside and lauryl glucoside sold, for example, by Henkel under the respective names PLANTAREN 2000 and PLANTAREN 1200, the cetostearyl glucoside optionally as a mixture with cetostearyl alcohol, sold, for example, under the name MONTANOV 68 by Seppic, under the name TEGO-CARE CG90 by Goldschmidt and under the name EMULGADE KE3302 by Henkel, and also the arachidyl glucoside, for example in the form of a mixture of arachidyl and behenyl alcohols and of arachidyl glucoside, sold under the name MONTANOV 202 by Seppic.

Use is more particularly made, as a nonionic surfactant of this type, of sucrose monostearate, sucrose distearate, sucrose tristearate and mixtures thereof, the distearate of methylglucose and polyglycerol-3 and alkyl polyglucosides.

4—The glycerol fatty esters, which can be used as nonionic surfactants that are solid at a temperature equal to 45° C., may especially be chosen from the group comprising esters formed from at least one acid consisting of a saturated linear alkyl chain having from 16 to 22 carbon atoms and from 1 to 10 glycerol units. It is possible to use one or more of these glycerol fatty esters.

These esters may especially be chosen from stearates, behenates, arachidates, palmitates and mixtures thereof. Preferably, stearates and palmitates are used.

Mention may be made, by way of example of a surfactant that can be used in the nanoemulsion of the invention, of decaglycerol (10 glycerol units) monostearate, distearate, tristearate and pentastearate (CTFA names: Polyglyceryl-10 stearate, Polyglyceryl-10 distearate, Polyglyceryl-10 tristearate, Polyglyceryl-10 pentastearate) such as the products sold under the respective names NIKKOL DECAGLYN 1-S, 2-S, 3-S and 5-S by Nikko, and diglycerol monostearate (CTFA name: Polyglyceryl-2 stearate) such as the product sold by Nikko under the name NIKKOL DGMS.

The sorbitan fatty esters, which can be used as nonionic surfactants that are solid at a temperature less than or equal to 45° C., are especially chosen from the group comprising C₁₆C₂₂ fatty acid and sorbitan esters and C₁₆-C₂₂ fatty acid and oxyethylenated sorbitan esters. They are formed from at least one fatty acid consisting of at least one saturated linear alkyl chain, respectively having from 16 to 22 carbon atoms, and from sorbitol or ethoxylated sorbitol. The oxyethylenated esters generally comprise from 1 to 100 ethylene oxide units and preferably from 2 to 40 ethylene oxide (EO) units.

These esters may especially be chosen from stearates, behenates, arachidates, palmitates and mixtures thereof. Preferably stearates and palmitates are used.

Mention may be made, by way of example of sorbitan fatty ester and oxyethylenated sorbitan fatty ester, which can be used in the nanoemulsion of the invention, of sorbitan monostearate (CTFA name: Sorbitan stearate) sold by ICI under the name SPAN 60, sorbitan monopalmitate (CTFA name: Sorbitan palmitate) sold by ICI under the name SPAN 40, sorbitan 20 EO tristearate (CTFA name: Polysorbate 65) sold by ICI under the name TWEEN 65.

The ethoxylated fatty ethers that are solid at a temperature less than or equal to 45° C. are preferably ethers formed from 1 to 100 ethylene oxide units and from at least one fatty alcohol chain having from 16 to 22 carbon atoms. The fatty chain of the ethers may especially be chosen from behenyl, arachidyl, stearyl and cetyl units, and mixtures thereof such as the cetearyl unit. By way of example of ethoxylated fatty ethers, mention may be made of behenyl alcohol ethers comprising 5, 10, 20 and 30 ethylene oxide units (CTFA name: Beheneth-5, Beheneth-10, Beheneth-20, Beheneth-30), such as the products sold under the names NIKKOL BB5, BB10, BB20, BB30 by Nikko, and the stearyl alcohol ether comprising 2 ethylene oxide units (CTFA name: Steareth-2), such as the product sold under the name BRIJ 72 by ICI.

The ethoxylated fatty esters that are solid at a temperature less than or equal to 45° C. are esters formed from 1 to 100 ethylene oxide units and from at least one fatty acid chain having from 16 to 22 carbon atoms. The fatty chain of the esters may especially be chosen from the stearate, behenate, arachidate and palmitate units, and mixtures thereof. By way of example of ethoxylated fatty esters, mention may be made of the stearic acid ester comprising 40 ethylene oxide units, such as the product sold under the name MYRJ 52 (CTFA name: PEG-40 stearate) by ICI and also the behenic acid ester comprising 8 ethylene oxide units (CTFA name: PEG-8 behenate), such as the product sold under the name COMPRITOL HD5 ATO by Gattefosse.

5—The ethylene oxide and propylene oxide block copolymers that can be used as nonionic surfactants may especially be chosen from the block copolymers of formula (V):

HO(C₂H₄O)ₓ(C₃H₆O)_{y}(C₂H₄O)ₙH (V)

in which x, y and z are integers such that x+z ranges from 2 to 100 and y ranges from 14 to 60, and mixtures thereof, and more particularly from block copolymers of formula (V) having an HLB ranging from 2 to 16.

These block copolymers may especially be chosen from the polyethylene glycol/polypropylene glycol/polyethylene glycol triblock polycondensates sold under the names SYNPERONIC such as SYNPERONIC® PE/L81 (INCI name: POLOXAMER 231) of formula (V) with x=z=6, y=39 (HLB 2); SYNPERONIC® PE/L92 (INCI name: POLOXAMER 282) of formula (V) with x=z=10, y=47 (HLB 6) by Uniqema.

As nonionic surfactants, mention may also be made of the mixtures of nonionic surfactants described in document EP-A-705 593 incorporated here for reference.

Among the nonionic surfactants, use may in particular be made of:
PEG 400 isostearate or PEG-8 isostearate (comprising 8 moles of ethylene oxide);
diglyceryl isostearate;
polyglycerol monolaurate having 2 glycerol units and polyglycerol stearates having 10 glycerol units;
sorbitan oleate;
sorbitan isostearate; and
mixtures thereof.

The anionic surfactants which may be used in the compositions of the invention are preferably chosen from:
the alkali metal salts of dicetyl and dimyristyl phosphate;
the alkali metal salts of cholesterol sulphate;
the alkali metal salts of cholesterol phosphate;
lipoamino acids and their salts such as monosodium and disodium acylglutamates such as the disodium salt of N-stearoyl-L-glutamic acid sold under the name ACYLGLUTAMATE HS21 by Ajinomoto;
the sodium salts of phosphatidic acid;
phospholipids;
alkylsulphonic derivatives especially of formula (VI):

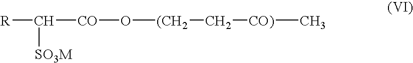

in which R represents C₁₆-C₂₂ alkyl radicals, in particular the C₁₆H₃₃ and C₁₈H₃₇ radicals taken as a mixture or separately and M is an alkali or alkaline-earth metal such as sodium; and
mixtures thereof.

The cationic surfactants which may be used in the compositions of the invention are preferably chosen from the group formed by quaternary ammonium salts, fatty amines and their salts.

The quaternary ammonium salts are, for example:
those which have the general formula (VII) below:

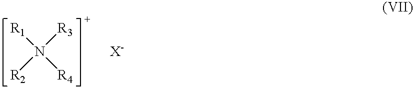

in which the radicals R₁ to R₄, which may be identical or different, represent a linear or branched aliphatic radical having from 1 to 30 carbon atoms, or an aromatic radical such as an aryl or alkylaryl radical. The aliphatic radicals may comprise heteroatoms such as, in particular, oxygen, nitrogen, sulphur or halogens. The aliphatic radicals are, for example, chosen from alkyl, alkoxy, polyoxy(C₂-C₆)alkylene, alkylamide, (C₁₂-C₂₂)alkylamido(C₂-C₆)alkyl, (C₁₂-C₂₂)alkyl acetate or hydroxyalkyl radicals, having around 1 to 30 carbon atoms; X is an anion chosen from the group of halides, phosphates, acetates, lactates, (C₂-C₆)alkyl sulphates, alkylsulphonates or alkylarylsulphonates;

Preferred from among the quaternary ammonium salts of formula (VII) are, on the one hand, tetraalkylammonium chlorides such as, for example, dialkyldimethylammonium or alkyltrimethylammonium chlorides, in which the alkyl radical has around 12 to 22 carbon atoms, in particular behenyltrimethylammonium, distearyldimethylammonium, cetyltrimethylammonium, benzyldimethylstearylammonium chlorides or else, on the other hand, stearamidopropyldimethyl(myristyl acetate)ammonium chloride sold under the name CERAPHYL 70 by Van Dyk. Behenyltrimethylammonium chloride is the most particularly preferred quaternary ammonium salt.
Quaternary ammonium salts of imidazolinium, such as for example that of formula (VIII) below:

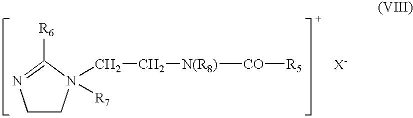

in which R₅ represents an alkenyl or alkyl radical having from 8 to 30 carbon atoms, for example derived from tallow fatty acids, R₆ represents a hydrogen atom, a C₁-C₄ alkyl radical or an alkenyl or alkyl radical having from 8 to 30 carbon atoms, R₇ represents a C₁-C₄ alkyl radical, R₈ represents a hydrogen atom, a C₁-C₄ alkyl radical, X is an anion chosen from the group of halides, phosphates, acetates, lactates, alkylsulphates, alkylsulphonates or alkylarylsulphonates. Preferably, R₅ and R₆ denote a mixture of alkenyl or alkyl radicals having from 12 to 21 carbon atoms, for example derived from tallow fatty acids, R₇ denotes methyl, R₈ denotes hydrogen. Such a product is, for example, sold under the name REWOQUAT W 75 by Rewo;
quaternary diammonium salts of formula (IX):

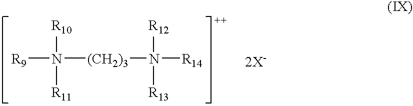

in which R₉ denotes an aliphatic radical having around 16 to 30 carbon atoms, R₁₀, R₁₁, R₁₂, R₁₃ and R₁₄, which are identical or different, are chosen from hydrogen or an alkyl radical having from 1 to 4 carbon atoms, and X is an anion chosen from the group of halides, acetates, phosphates, nitrates and methylsulphates. Such quaternary diammonium salts comprise, in particular, propanetallowediammonium dichloride;
quaternary ammonium salts containing at least one ester functional group.

The quaternary ammonium salts containing at least one ester functional group that can be used according to the invention are, for example, those of formula X below:

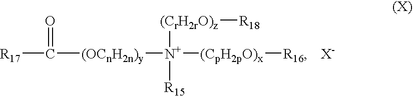

in which:
R₁₅ is chosen from C₁-C₆ alkyl radicals and C₁-C₆ hydroxyalkyl or dihydroxyalkyl radicals;
R₁₆ is chosen from:
radical R₁₉

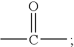

the saturated or unsaturated, linear or branched C₁-C₂₂ hydrocarbon-based radicals R₂₀; and
a hydrogen atom;
R₁₈ is chosen from:
radical R₂₁

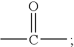

the saturated or unsaturated, linear or branched C₁-C₆ hydrocarbon-based radicals R₂₂; and
a hydrogen atom;
R₁₇, R₁₉ and R₂₁, which are identical or different, are chosen from saturated or unsaturated, linear or branched C₇-C₂₁ hydrocarbon-based radicals;
n, p and r, which are identical or different, are integers ranging from 2 to 6;
y is an integer ranging from 1 to 10;
x and z, which are identical or different, are integers ranging from 0 to 10; and
X⁻ is an organic or inorganic single or complex anion,

on condition that the sum x+y+z is equal to 1 to 15, that when x is equal to 0 then R₁₆ denotes R₂₀ and that when z is equal to 0 then R₁₈ denotes R₂₂.

The alkyl radicals R₁₅ may be linear or branched and more particularly linear.

Preferably, R₁₅ denotes a methyl, ethyl, hydroxyethyl or dihydroxypropyl radical, and more particularly a methyl or ethyl radical.

Advantageously, the sum x+y+z is equal to 1 to 10.

When R₁₆ is a hydrocarbon-based radical R₂₀, it may be long and have from 12 to 22 carbon atoms or short and have from 1 to 3 carbon atoms.

When R₁₈ is a hydrocarbon-based radical R₂₂, it preferably has from 1 to 3 carbon atoms.

Advantageously, R₁₇, R₁₉ and R₂₁, which are identical or different, are chosen from saturated or unsaturated, linear or branched C₁₁-C₂₁ hydrocarbon-based radicals, and more particularly from saturated or unsaturated, linear or branched C₁₁-C₂₁ alkyl and alkenyl radicals.

Preferably, x and z, which are identical or different, are equal to 0 or 1.

Advantageously, y is equal to 1.

Preferably, n, p and r, which are identical or different, are equal to 2 or 3 and even more particularly are equal to 2.

In the formula X, the anion X⁻ is preferably a halide (chloride, bromide or iodide) or an alkylsulphate, more particularly methylsulphate. It is possible however to use methanesulphonate, phosphate, nitrate, tosylate, an anion derived from an organic acid such as the acetate or lactate or any other anion compatible with the ammonium having an ester functional group. The anion X⁻ is even more particularly the chloride or methylsulphate.

Use is more particularly made of the ammonium salts of formula X in which:
R₁₅ denotes a methyl or ethyl radical;
x and y are equal to 1;
z is equal to 0 or 1;
n, p and r are equal to 2;
R₁₆ is chosen from:
radical R₁₉

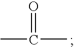

methyl, ethyl or C₁₄-C₂₂ hydrocarbon-based radicals; and
a hydrogen atom;
R₁₈ is chosen from:
radical R₂₁ and

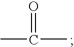

a hydrogen atom.

R₁₇, R₁₉ and R₂₁, which are identical or different, are chosen from saturated or unsaturated, linear or branched C₁₃-C₁₇ hydrocarbon-based radicals and preferably from saturated or unsaturated, linear or branched C₁₃-C₁₇ alkyl and alkenyl radicals.

Advantageously, the hydrocarbon-based radicals are linear.

Mention may for example be made, as compounds of formula X, of the salts (chloride or methylsulphate in particular) of diacyloxyethyldimethylammonium, diacyloxyethylhydroxyethylmethylammonium, monoacyloxyethyldihydroxyethylmethylammonium, triacyloxyethylmethylammonium, monoacyloxyethylhydroxyethyldimethylammonium and mixtures thereof. The acyl radicals preferably have 14 to 18 carbon atoms and more particularly come from a vegetable oil such as palm oil or sunflower oil. When the compound contains several acyl radicals, the latter may be identical or different. These products are obtained, for example, by direct esterification of the triethanolamine, triisopropanolamine, alkyldiethanolamine or alkyldiisopropanolamine, optionally oxyalkylenated, on fatty acids or on mixtures of fatty acids of plant or animal origin or by transesterification of their methyl esters. This esterification is followed by quaternization using an alkylating agent such as an alkyl (preferably methyl or ethyl) halide, dialkyl (methyl or ethyl preferably) sulphate, methyl methanesulphonate, methyl paratoluene-sulphonate and glycol or glycerol chlorohydrin.

Such compounds are, for example, sold under the names DEHYQUART by Henkel, STEPANQUAT by Stepan, NOXAMIUM by Ceca, REWOQUAT WE 18 by Rewo-Witco.

When it contains ammonium salts, the composition according to the invention preferably contains a mixture of quaternary ammonium monoester, diester and triester salts, with a weight majority of diester salts.

As a mixture of ammonium salts, it is possible to use, for example, the mixture containing 15 to 30% by weight of acyloxyethyldihydroxyethylmethylammonium methyl-sulphate, 45 to 60% of diacyloxyethylhydroxyethylmethylammonium methylsulphate and 15 to 30% of triacyloxyethylmethylammonium methylsulphate, the acyl radicals having from 14 to 18 carbon atoms and coming from palm oil, optionally partially hydrogenated.

It is also possible to use ammonium salts containing at least one ester functional group described in patents U.S. Pat. No. 4,874,554 and U.S. Pat. No. 4,137,180.

As surfactants, it is also possible to use emulsifiers based on polyolefins having a polar part, which are especially described, for example, in Application EP-A-1172 089.

The polyolefins having polar part(s) used in the composition of the invention are composed of an apolar polyolefin part and of at least one polar part. They may have a block or comb structure.

It is possible to use polyolefins having a polyoxyethylenated polar part, for example chosen from polyisoprene/polyoxyethylene diblock polymers, poly(ethylene-co-propylene)/polyoxyethylene polymers and mixtures thereof. These polymers are described in the publication by Allgaier, Poppe, Willner, Richter (Macromolecules, 1997, vol. 30, p. 1582-1586).

It is possible to use polyolefins having a succinic acid or anhydride polar part chosen, in particular, from the polyolefin derivatives of succinic acid or anhydride described in patents U.S. Pat. No. 4,234,435, U.S. Pat. No. 4,708,753, U.S. Pat. No. 5,129,972, U.S. Pat. No. 4,931,110, GB-A-2 156 799, U.S. Pat. No. 4,877,756 and U.S. Pat. No. 4,919,179.

It is possible to use polyolefins having modified (esterified or amidified) succinic ends and their preparation method is described, in particular, in document U.S. Pat. No. 4,708,753. Preferably, polyolefins having esterified succinic ends are used.

As polyolefins having succinic ends, mention may especially be made of polyisobutylenes having esterified succinic ends, especially esterified by diethanolamine, and their salts, especially the salts of diethanolamine, such as the products sold under the names LUBRIZOL® 2724, LUBRIZOL® 2722 and LUBRIZOL® 5603 by Lubrizol.

Another example of a polyolefin having a polar part that can be used in the invention is the reaction product of maleic anhydride with polyisobutylene, such as the products sold under the names GLISSOPAL (GLISSOPAL 2300, 1300 and 1000) (INCI name: polyisobutene) by BASF.

The polyolefin having a polar part that is particularly preferred is a reaction product of polyisobutenyl succinic anhydride with diethylethanolamine, thus forming a diethylethanolamine salt of 2-(N,N-diethyl)aminoethyl polybutene succinate. This product is sold, for example, under the name LUBRIZOL® 5603 by Lubrizol. The INCI name of this product is: Hydroxyethyldiethonium Polyisobutenyl Triethylaminosuccinate (and) Diethyl ethanolamine.

Another polyolefin having a polar part that is particularly preferred is an ester of triethanolamine diethanolaminoethyl polyisobutenyl succinate. This product is sold, for example, under the name CHEMCCINATE® 2000 by Chemron.

As a polyolefin having a polar part, it is also possible to use an ester of glyceryl polyisobutenyl succinate, especially that sold under the name CHEMCCINATE® 1000 AF by Chemron.

As surfactants, it is also possible to use emulsifying silicone elastomers such as those sold under the names KSG-210, KSG-310, KSG-320, KSG-330, KSG-440, KSG-710, KSG-KSG-830, KSG-840 by Shin-Etsu.

It is also possible to use polymers of isophthalic acid or sulphoisophthalic acid, and in particular the phthalate/sulphoisophthalate/glycol copolymers (for example diethyleneglycol/phthalate/isophthalate/1,4-cyclohexanedimethanol copolymer) sold under the names EASTMAN AQ POLYMER (AQ35S, AQ38S, AQ55S, AQ48 Ultra) by Eastman Chemical. Such surfactants are described in Application EP-A-864 320.

It is also possible to use hydrophobic polymers of 2-acrylamido-2-methyl-propanesulphonic acid (AMPS) such as the copolymer of AMPS and ethoxylated C₁₂-C₁₄ alcohol methacrylate (non-crosslinked copolymer obtained from GENAPOL LA-070 and AMPS) (CTFA name: Ammonium Acryloyldimethyltaurate/Laureth-7 Methacrylate Copolymer) sold under the name ARISTOFLEX LNC by Clariant, and the copolymer of AMPS and ethoxylated (25 EO) stearyl methacrylate (copolymer crosslinked by trimethylolpropane triacrylate, obtained from GENAPOL T-250 and AMPS) (CTFA name: Ammonium Acryloyldimethyltaurate/Steareth-25 Methacrylate Crosspolymer) sold under the name ARISTOFLEX HMS by Clariant. These polymers are especially described in Application EP-A-1 661 550.

Of course, a person skilled in the art will be sure to choose this or these optional additional compounds, and/or their amount, so that the advantageous properties of the composition corresponding to the invention are not, or are not substantially, impaired by the envisaged addition.

The compositions according to the invention may be independently in the form of a suspension, dispersion, solution, gel, emulsion, especially an oil-in-water (O/W), wax-in-water or water-in-oil (W/O) or multiple (W/O/W or polyol/O/W or O/W/O) emulsion, in the form of a cream, paste, foam, dispersion of vesicles, especially ionic or nonionic lipids, two-phase or multiphase lotion, paste, especially a soft paste.

The compositions according to the invention or used for the method according to the invention can be in the form of a composition for protection, treatment or care for the face, the hands, the feet, for the principal anatomic folds or for the body (for example day cream, night cream, make-up-remover cream, sunscreen composition, body milk for protection or care, after-sun milks, skin-care lotion, gel or mousse, artificial tanning composition); an after-shave composition.

They may also be used for make-up of the skin, lips, eyelashes and/or nails depending on the nature of the ingredients used.

In particular, the compositions according to the invention may be independently in the form of a foundation, a product for the lips, especially a lipstick, a concealer or eye contour product, eyeliner, mascara, eyeshadow, make-up product for the body or else a product for dyeing the skin.

According to one embodiment, the compositions are compositions for coating the skin of the body or of the face, more particularly compositions for the make-up or care of the skin of the body or of the face such as for example foundations or body make-up compositions.

A person skilled in the art will be able to select the appropriate galenical form, as well as its method of preparation, on the basis of his general knowledge, taking into account, on the one hand, the nature of the constituents used, notably their solubility in the support, and, on the other hand, the application envisaged for each composition.

The invention is illustrated in more detail by the examples described below. Unless stated otherwise, the quantities shown are expressed as percentage by weight.

In the examples of compositions described hereunder, the combination of the following mixtures A and B produced by the company Dow Corning is used as compounds X and Y:

Mixture A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

Mixture B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

### EXAMPLE 1

### Preparation of Nanocapsules of a Compound X or Y According to the Invention

Organic phase:

| | | |
|---|---|---|
| Mixture B | 3.62 | g |
| Polycaprolactone (Capa ® 6100, Solvay) | 0.48 | g |
| Dimethicone copolyol (DC SH 3773 M, Dow Corning | 0.24 | g |
| Dichloromethane | 16 | ml |

Aqueous phase:

| | | |
|---|---|---|
| Disodium salt of N-stearoyl-L-glutamic acid | 0.12 | g |
| (AMISOFT HS21P from Ajinomoto) | | |
| Distilled water | 24.14 | g |

An organic/water pre-emulsion was produced using means known to a person skilled in the art for producing emulsions such as, for example, by using an Ultra Turrax T25 stirrer for 5 min at 11 000 rpm.

This pre-emulsion was then homogenized, using a OBL20 type high-pressure homogenizer from Niro Soavi, 2 times at 600 bar to obtain capsules of less than 1 μm.

The dichloromethane was then evaporated using a rotary evaporator under reduced pressure.

Thus, a suspension of nanocapsules containing 15% of mixture Y representing one of the compounds X or Y according to the invention was obtained. The control of the size of the nanocapsules was carried out using a laser particle size analyzer by light scattering. The average size of the nanocapsules was 256 nm.

These capsules were perfectly stable for 2 months at +4° C., ambient temperature and 45° C.

### EXAMPLE 2

### Preparation of Nanocapsules of the Compound Additional to that of Example 1

Organic phase:

| | | |
|---|---|---|
| Mixture A | 3.62 | g |
| Polycaprolactone (Capa ® 6100, Solvay) | 0.48 | g |
| Dimethicone copolyol (DC SH 3773 M, Dow Corning | 0.24 | g |
| Dichloromethane | 16 | ml |

Aqueous phase:

| | | |
|---|---|---|
| Disodium salt of N-stearoyl-L-glutamic acid | 0.12 | g |
| (AMISOFT HS21P from Ajinomoto) | | |
| Distilled water | 24.14 | g |

The same procedure as that described in Example 1 was followed. A suspension of 260 nm nanocapsules was obtained.

These capsules were perfectly stable for 2 months at +4° C., ambient temperature and 45° C.

### EXAMPLE 3

### Preparation of a Composition According to the Invention

50 g of the suspension obtained in Example 1 was mixed with 50 g of the suspension obtained in Example 2.

The mixture remained liquid, no change was observed over time.

When a film of the preceding mixture is formed on a glass plate, the crosslinking of the two reactive silicones is observed during the evaporation of the water. An opalescent, cohesive and peelable film is formed.

### EXAMPLE 4

### Day Cream for the Face, Intended for Dry Skin

Phase A1:

| | |
|---|---|
| Sucrose distearate sold by Sterinerie Dubois | 2.00% |
| Oxyethylenated sorbitan stearate having 4 mols of ethylene | 1.40% |
| oxide sold under the name TWEEN 61 ® by ICI | |
| Stearic acid | 0.75% |
| Codex liquid petroleum | 2.10% |
| Avocado oil | 4.50% |
| Jojoba oil | 4.10% |
| Volatile silicone oil | 3.70% |
| Vitamin E acetate | 0.50% |
| D-α-tocopherol sold by Henkel under the name | 0.30% |
| COPHEROL 1300 ® | |

Phase A2:

| | |
|---|---|
| Fragrance | 0.30% |
| Propylparaben | 0.10% |

Phase B:

| | |
|---|---|
| Glycerol | 5.00% |
| Methylparaben | 0.30% |
| Triethanolamine | 0.40% |
| Demineralized water | qs for 100% |

Phase C:

| | |
|---|---|
| Mixture of carboxyvinyl polymers sold under the name | 0.30% |
| CARBOPOL 940 ® by Noveon | |
| Demineralized water | 9.70% |

Phase D:

| | |
|---|---|
| Aqueous suspension of capsules from Example 1 | 10.00% |
| Aqueous suspension of capsules from Example 2 | 10.00% |

Procedure

The oily phase A1 and the aqueous phase B were heated separately to a temperature of 80° C.

While stirring at 4000 rpm using a Mortiz Turbo Lab 2100 homogenizer, phase B was poured over phase A1 and these stirring and temperature conditions were maintained for 30 minutes.

The mixture was then introduced into a Soavi OBL-type high-pressure homogenizer set at a pressure of 500 bar for 3 consecutive passes. The oily phase A2 was then added to the emulsion and the whole mixture was stirred using the Turbo Lab 2100 at a speed of 3000 rpm for 10 min. Phase C was added to this emulsion A1+B+A2 and the whole mixture was stirred using a Rayneri homogenizer equipped with a deflocculating type impeller at a speed of 2500 rpm for 30 min at ambient temperature. Phase D was then added under reduced stirring.

An emulsion was obtained of which the application to the skin caused the rupture of the capsules and the thus released encapsulated compounds reacted to form a polymer film on the skin.

### EXAMPLE 5

### Sun Composition

Phase A:

| | |
|---|---|
| Diglycol/cyclohexane dimethanol/isophthalate/sulpho- | 2.00% |
| isophthalate copolymer (AQ38S from Eastman | |
| Chemical) | |
| Glycerol | 5.00% |
| Preservatives | 1.20% |
| Sequestrants | 0.10% |
| Demineralized water | 34.70% |

Phase B:

| | |
|---|---|
| 2-Ethylhexyl 2-cyano-3,3-diphenylacrylate | 10.00% |
| 4-tert-Butyl-4′-methoxydibenzoylmethane (PARSOL | 2.00% |
| 1789 sold by Givaudan) | |
| Cyclomethicone | 4.00% |
| Liquid jojoba wax (Flora Tech) | 4.00% |

Phase C:

| | |
|---|---|
| Xanthan gum | 0.50% |
| Demineralized water | qs for 100.00% |

Phase D:

| | |
|---|---|
| Aqueous suspension of capsules from Example 1 | 10.00% |
| Aqueous suspension of capsules from Example 2 | 10.00% |

Procedure

The constituents of phase A were mixed and the mixture was heated at 70° C. with magnetic stirring until complete dispersion of the polymer, then the solution was cooled to ambient temperature. Furthermore, the phase B was prepared.

Phase A was introduced into phase B with rapid stirring. The emulsion was homogenized under a pressure of 600 bar (2 to 4 passes), bringing the emulsion to ambient temperature between each pass.

Phases C and D were then added using a Rayneri homogenizer equipped with a deflocculating type impeller.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic composition intended for the care and/or make-up of keratinous substance(s) comprising, in a physiologically acceptable medium, at least one compound X, one compound Y, and at least one catalyst, with at least one of the compounds X or Y being a polyorganosiloxane, and said compounds X and Y being capable of reacting together by a hydrosilylation reaction in the presence of a catalyst, with at least one compound from the compounds X and Y being present in said composition in an encapsulated form, said catalyst being combined with at least one of the encapsulated compounds X or Y.

2. Composition according to claim 1, wherein the compounds X and Y are both present in separated encapsulated forms.

3. Composition according to claim 1, wherein the encapsulated form is a core/shell type nanocapsule of which the core of lipophilic nature is formed completely or partly either from at least one compound X, or from at least one compound Y, or from an oil containing at least one catalyst, said catalyst being encapsulated with the compound X or the compound Y.

4. Composition according to claim 3, wherein the shell of the nanocapsules is of polymeric nature.

5. Composition according to claim 4, wherein the shell is of polymeric nature which is not crosslinked, water-insoluble and insoluble in the core of said nanocapsules.

6. Composition according to claim 4, wherein the polymer of the shell has a melting point below 100° C.

7. Composition according to claim 4, wherein the shell of the nanocapsules comprises at least one polymer chosen from the group consisting of
C₂-C₁₂ alkyl cyanoacrylate polymers;
poly-L-lactides, poly-DL-lactides, polyglycolides and the corresponding copolymers;
polycaprolactones;
polymers of 3-hydroxybutyric acid;
copolymers of vinyl chloride and vinyl acetate;
polyvinyl acetophthalate;
cellulose acetophthalate;
polyvinylpyrrolidone-vinyl acetate copolymer;
polyethylenevinyl acetates;
copolymers of methacrylic acid and methacrylic ester;
polyacrylonitriles;
polyacrylamides;
polyethylene glycols;
poly(C₁ to C₄ hydroxyalkyl methacrylate);
cellulose derivatives;
polystyrene and its copolymers;
styrene alkylalcohol oligomers;
terpolymers of ethylene, vinyl acetate and maleic anhydride;
polyamides;
polyethylenes;
polypropylenes;
organopolysiloxanes;
poly(alkylene adipate);
polyol polyesters;
polysilsesquioxane silicone polymers;
dendritic polyesters with a hydroxyl terminal function; and
mixtures thereof.

8. Composition according to claim 4, wherein the polymer of the shell of the nanocapsules is chosen from:
polycaprolactones;
polyvinyl acetophthalate;
cellulose acetophthalate;
methacrylic acid and methacrylic ester copolymers;
derivatives of cellulose;
polystyrene and its copolymers;
polyamides;
polyorganosiloxanes;
poly(alkylene adipate); and
mixtures thereof.

9. Composition according to claim 4, wherein the shell of the nanocapsules comprises at least one polymer chosen from polycaprolactones.

10. Composition according to claim 3, wherein the nanocapsules have a diameter less than 1 μm.

11. Composition according to claim 1, wherein compound X is selected from silicone compounds comprising at least two unsaturated aliphatic groups.

12. Composition according to claim 11, wherein compound X is a polyorganosiloxane comprising a silicone main chain whose unsaturated aliphatic groups are pendent from the main chain (side group) or located at the ends of the main chain of the compound (end group).

13. Composition according to claim 12, wherein compound X bears at least one polar group.

14. Composition according to claim 1, wherein compound X is selected from the polyorganosiloxanes comprising at least two unsaturated aliphatic groups each attached to a silicon atom.

15. Composition according to claim 1, wherein compound X is selected from the polyorganosiloxanes containing siloxane units of formula:
$${R}_{m} {R}^{′} {SiO}_{\frac{( 3 - m )}{2}} \qquad ( I )$$
in which:
R represents a linear or cyclic, monovalent hydrocarbon group, having from 1 to 30 carbon atoms,
m is equal to 1 or 2 and
R′ represents:
an unsaturated aliphatic hydrocarbon group having from 2 to 10 carbon atoms or an unsaturated cyclic hydrocarbon group having from 5 to 8 carbon atoms.

16. Composition according to claim 15, wherein the polyorganosiloxane of formula (I) is such that R′ represents a vinyl group or a group —R″-CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms.

17. Composition according to claim 15, wherein R represents an alkyl radical having from 1 to 10 carbon atoms or alternatively a phenyl group, and R′ is a vinyl group.

18. Composition according to claim 12, wherein the polyorganosiloxanes additionally comprise units of formula:
$${R}_{n} {SiO}_{\frac{( 4 - n )}{2}} \qquad ( II )$$
in which R represents a linear or cyclic, monovalent hydrocarbon group, having from 1 to 30 carbon atoms, and n is equal to 1, 2 or 3.

19. Composition according to claim 1, wherein compound X is selected from organic oligomers or polymers, hybrid organic/silicone oligomers or polymers, said oligomers or polymers bearing at least 2 unsaturated reactive aliphatic groups.

20. Composition according to claim 1, wherein compound Y comprises at least two free Si—H groups.

21. Composition according to claim 1, wherein compound Y is selected from the polyorganosiloxanes comprising at least one alkylhydrogenosiloxane unit with the following formula:
$${R}_{p} {HSiO}_{\frac{( 3 - p )}{2}} \qquad ( III )$$
in which:
R represents a linear or cyclic, monovalent hydrocarbon group, having from 1 to 30 carbon atoms or a phenyl group, and p is equal to 1 or 2.

22. Composition according to claim 21, wherein compound Y is such that the radicals R represent a C₁-C₁₀ alkyl group.

23. Composition according to claim 20, wherein Y is a polyorganosiloxane comprising at least two alkylhydrogenosiloxane units of formula —(H₃C)(H)Si—O—.

24. Composition according to claim 1, wherein the hydrosilylation is performed in the presence of a catalyst based on platinum or tin.

25. Composition according to claim 24, wherein the catalyst is present at a content ranging from 0.0001 to 20 wt. % relative to the total weight of the composition containing it.

26. Composition according to claim 1, wherein compound X is a polydimethylsiloxane with vinylic end groups and compound Y is a polymethylhydrogenosiloxane.

27. Composition according to claim 1, wherein the catalyst is encapsulated with a compound X chosen from polyorganosiloxanes comprising at least two unsaturated aliphatic groups.

28. Composition according to claim 1, wherein compound X bears at least one polar group that is able to form a hydrogen bond with keratinous substances.

29. Composition according to claim 1, furthermore comprising at least one filler selected from silica or surface-treated silica.

30. Composition according to claim 1, wherein compound X has a weight-average molecular weight (Mw) in the range from 150 to 1 000 000.

31. Composition according to claim 1, wherein compound Y has a weight-average molecular weight (Mw) in the range from 200 to 1 000 000.

32. Composition according to claim 1, wherein compound X represents from 0.5 to 95 wt. % relative to the total weight of the composition.

33. Composition according to claim 1, wherein compound Y represents from 0.5 to 95 wt. % relative to the total weight of the composition.

34. Composition according to claim 1, wherein compounds X and Y are present in the composition in a molar ratio X/Y in the range from 0.05 to 20.

35. Composition according to claim 1, comprising a liquid fatty phase.

36. Composition according to claim 35, wherein the liquid fatty phase comprises at least one oil.

37. Composition according to claim 36, wherein the oil or oils are present at a content ranging from 1 to 90 wt. % relative to the total weight of the composition.

38. Composition according to claim 1, comprising, in addition, at least one compound chosen from vitamins, thickeners, gelling agents, trace elements, softeners, sequestrants, fragrances, acidifying or basifying agents, preservatives, fragrances, sunscreens, surfactants, antioxidants, fibres, fillers, dyes, film-forming polymers, waxes, cosmetic active agents such as bactericidal or antiperspirant active agents, neutralizers, emollients, moisturizers and mixtures thereof.

39. Composition according to claim 1, wherein it is a composition for coating the skin of the body or face.

40. Cosmetic coating method for the care and/or make-up of keratinous substance(s) comprising at least the application to said keratinous substance of a mixture of compounds X and Y capable of reacting together by a hydrosilylation reaction in the presence of a catalyst, said mixture being derived from a cosmetic composition comprising, in a physiologically acceptable medium, at least one such compound X, one such compound Y and at least one catalyst and in which at least one compound from the compounds X, Y and the catalyst is present in said composition in an encapsulated form.

41. Method according to claim 40, wherein the mixture is obtained either extemporaneously before application to said keratinous substance, or at the time that said composition is applied to said keratinous substance.

42. Method according to claim 40, wherein the composition comprises, in a physiologically acceptable medium, at least one compound X, one compound Y, and at least one catalyst, with at least one of the compounds X or Y being a polyorganosiloxane, and said compounds X and Y being capable of reacting together by a hydrosilylation reaction in the presence of a catalyst, with at least one compound from the compounds X and Y being present in said composition in an encapsulated form, said catalyst being combined with at least one of the encapsulated compounds X or Y.

43. A method comprising using a composition comprising an encapsulated form of at least one compound X, an encapsulated form of at least one compound Y and/or an encapsulated form of at least one catalyst for the care and/or make-up of keratinous substance(s) comprising applying said composition to said keratinous substance, in a physiologically acceptable medium, to form, after application and drying on said keratinous substance, a polymeric film derived from the reaction of a compound X and a compound Y according to a reaction of hydrosilylation type in the presence of a catalyst, with at least one of the compounds X or Y being a polyorganosiloxane.

44. Method according to claim 43, wherein the composition comprises, in a physiologically acceptable medium, at least one compound X, one compound Y, and at least one catalyst, with at least one of the compounds X or Y being a polyorganosiloxane, and said compounds X and Y being capable of reacting together by a hydrosilylation reaction in the presence of a catalyst, with at least one compound from the compounds X and Y being present in said composition in an encapsulated form, said catalyst being combined with at least one of the encapsulated compounds X or Y.

45. Cosmetic composition intended for the care and/or make-up of keratinous substance(s) comprising, in a physiologically acceptable medium, at least one compound X and one compound Y, and optionally a catalyst, with at least one of compounds X and Y being a polyorganosiloxane, and said compounds X and Y being capable of reacting together by a condensation reaction, with at least one compound from the compounds X, Y and the catalyst, when it is present, being present in said composition in an encapsulated form.
